(19) United States
(12) Patent Application Publication
(10) Pub. No.: US 2024/0268703 A1
(43) Pub. Date: Aug. 15, 2024

(54) SYSTEMS AND METHODS FOR MEDICAL IMAGING

(71) Applicant: SHANGHAI UNITED IMAGING HEALTHCARE CO., LTD., Shanghai (CN)

(72) Inventors: Qinhua HUANG, Shanghai (CN); Jianqiao CHEN, Shanghai (CN); Shiming HU, Shanghai (CN); Xiaochun XU, Shanghai (CN); Xiaoyue GU, Shanghai (CN); Shitao LIU, Shanghai (CN); Shengguo JIA, Shanghai (CN)

(73) Assignee: SHANGHAI UNITED IMAGING HEALTHCARE CO., LTD., Shanghai (CN)

(21) Appl. No.: 18/592,472

(22) Filed: Feb. 29, 2024

Related U.S. Application Data
(63) Continuation of application No. PCT/CN2022/116813, filed on Sep. 2, 2022.

(30) Foreign Application Priority Data
Sep. 2, 2021 (CN) 202122114423.4
Nov. 29, 2021 (CN) 202111435340.3
Dec. 31, 2021 (CN) 202111681148.2

(51) Int. Cl. A61B 5/08; A61B 5/00; A61B 5/11; A61B 5/113

(52) U.S. Cl. CPC A61B 5/0816; A61B 5/0064; A61B 5/1128; A61B 5/113; A61B 5/742

(57) ABSTRACT

Systems and methods for medical imaging may be provided. A respiratory amplitude of a respiratory motion of a subject during a medical scan may be determined based on a respiratory signal relating to the respiratory motion. The respiratory signal may be collected using a respiratory motion detector by emitting detecting signals toward a target region of the subject. Surface information of the target region may be obtained. The respiratory amplitude may be corrected based on the surface information of the target region.

### CROSS-REFERENCE TO RELATED APPLICATIONS

This application is a continuation of International Application No. PCT/CN2022/116813, filed on Sep. 2, 2022, which claims priority to Chinese Patent Application No. 202122114423.4, filed on Sep. 2, 2021, Chinese Patent Application No. 202111435340.3, filed on Nov. 29, 2021, and Chinese Patent Application No. 202111681148.2, filed on Dec. 31, 2021, the contents of each of which are hereby incorporated by reference.

### TECHNICAL FIELD

The present disclosure relates to medical technology, and in particular, to systems and methods for medical imaging.

### BACKGROUND

Medical imaging technology has been widely used for creating images of interior of a patient's body for, e.g., medical diagnosis and/or treatment purposes.

### SUMMARY

According to an aspect of the present disclosure, a system for medical imaging may be provided. The system may include at least one storage device including a set of instructions and at least one processor. The at least one processor may be configured to communicate with the at least one storage device. When executing the set of instructions, the at least one processor may be configured to direct the system to perform one or more of the following operations. The system may determine a respiratory amplitude of a respiratory motion of a subject during a medical scan based on a respiratory signal relating to the respiratory motion. The respiratory signal may be collected using a respiratory motion detector by emitting detecting signals toward a target region of the subject. The system may also obtain surface information of the target region. The system may further correct the respiratory amplitude based on the surface information of the target region.

In some embodiments, the corrected respiratory amplitude may reflect an intensity of the respiratory motion of the subject along a standard direction.

In some embodiments, to obtain surface information of the target region, the system may acquire a three-dimensional (3D) optical image of the subject using an image acquisition device. The system may determine the surface information of the target region based on the 3D optical image of the subject.

In some embodiments, to correct the respiratory amplitude based on the surface information of the target region, the system may determine a surface profile of the target region based on the surface information of the target region. The system may also divide the surface profile into a plurality of subsections. For each of the plurality of subsections, the system may determine a correction factor corresponding to the subsection. The system may further correct the respiratory amplitude of the subject based on the plurality of correction factors corresponding to the plurality of subsections.

In some embodiments, for each of the plurality of subsections, to determine a correction factor corresponding to the subsection, the system may obtain an installation angle of the respiratory motion detector relative to a reference direction. The system may also determine an included angle between the subsection and the reference direction. The system may further determine the correction factor corresponding to the subsection based on the installation angle and the included angle.

In some embodiments, the determining a respiratory amplitude of a respiratory motion of a subject may comprise determining a plurality of respiratory amplitudes of the respiratory motion at a plurality of time points during the medical scan based on the respiratory signal. The obtaining surface information of the target region may comprise obtaining sets of surface information of the target region. Each of the sets of surface information may correspond to one of the plurality of time points. The correcting the respiratory amplitude may comprise, for each of the plurality of time points, correcting the respiratory amplitude at the time point based on the surface information corresponding to the time point.

In some embodiments, the at least one processor may be further configured to direct the system to perform one or more of the following operations. The system may obtain scan data of the subject collected by medical scan. The system may further process the scan data of the subject based on the corrected respiratory amplitudes corresponding to the plurality of time points.

In some embodiments, the at least one processor may be configured to direct the system to perform one or more of the following operations. The system may determine motion data of the subject based on at least one of respiratory motion data or posture data. The respiratory motion data may include the corrected respiratory amplitude values corresponding to the plurality of time points and the posture data may be collected over a time period including the plurality of time points. The system may also determine whether the subject has an obvious motion in the time period based on the motion data of the subject. In response to determining that the subject has an obvious motion in the time period, the system may control a display device to perform a target operation.

In some embodiments, the display device may include a projector disposed in a scanning tunnel of a medical scanner that performs the medical scan.

In some embodiments, the projector may be configured to project a virtual character in a first status on an inside wall of the scanning tunnel. To control a display device to perform a target operation, the system may control the projector to change the projected virtual character from the first status to a second status.

In some embodiments, the at least one processor may be configured to direct the system to perform one or more of the following operations. The system may obtain a scout image of the subject collected by a scout scan. The scout scan may be performed on the subject before the medical scan. The system may perform foreign matter detection on the scout image of the subject using at least one foreign matter detection model. The system may further determine whether the medical scan can be started based on a result of the foreign matter detection.

In some embodiments, the at least one processor may be configured to direct the system to perform one or more of the following operations. In response to a result of the foreign matter detection that non-iatrogenic foreign matter is disposed on or within the subject, the system may generate first prompt information for requiring the subject to take off the non-iatrogenic foreign matter.

In some embodiments, the at least one processor may be configured to direct the system to perform one or more of the following operations. In response to a result of the foreign matter detection that iatrogenic foreign matter is disposed on or within the subject, the system may generate second prompt information for reminding that artifact correction needs to be performed on the medical scan.

According to another aspect of the present disclosure, a method for medical imaging may be provided. The method may include determining a respiratory amplitude of a respiratory motion of a subject during a medical scan based on a respiratory signal relating to the respiratory motion. The respiratory signal may be collected using a respiratory motion detector by emitting detecting signals toward a target region of the subject. The method may also include obtaining surface information of the target region. The method may further include correcting the respiratory amplitude based on the surface information of the target region.

According to yet another aspect of the present disclosure, a system for medical imaging may be provided. The system may include a determination module, an acquisition module, and a correction module. The determination module may be configured to determine a respiratory amplitude of a respiratory motion of a subject during a medical scan based on a respiratory signal relating to the respiratory motion. The respiratory signal may be collected using a respiratory motion detector by emitting detecting signals toward a target region of the subject. The acquisition module may be configured to obtain surface information of the target region. The correction module may be configured to correct the respiratory amplitude based on the surface information of the target region.

According to yet another aspect of the present disclosure, a non-transitory computer readable medium may be provided. The non-transitory computer readable medium may include at least one set of instructions for medical imaging. When executed by one or more processors of a computing device, the at least one set of instructions may cause the computing device to perform a method. The method may include determining a respiratory amplitude of a respiratory motion of a subject during a medical scan based on a respiratory signal relating to the respiratory motion. The respiratory signal may be collected using a respiratory motion detector by emitting detecting signals toward a target region of the subject. The method may also include obtaining surface information of the target region. The method may further include correcting the respiratory amplitude based on the surface information of the target region.

According to yet another aspect of the present disclosure, a device for medical imaging may be provided. The device may include at least one processor and at least one storage device for storing a set of instructions. When the set of instructions may be executed by the at least one processor, the device performs the method for medical imaging.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1 is a schematic diagram illustrating an exemplary medical imaging system 100 according to some embodiments of the present disclosure;

FIG. 2 is a block diagram illustrating exemplary processing device according to some embodiments of the present disclosure;

FIG. 3 is a schematic diagram illustrating an exemplary medical imaging process according to some embodiments of the present disclosure;

FIG. 4 is a flowchart illustrating an exemplary process 400 for correcting a respiratory amplitude of a respiratory motion of a subject during a medical scan according to some embodiments of the present disclosure;

FIG. 5 is a schematic diagram illustrating an exemplary installation positions of a respiratory motion detector and an image acquisition device in a medical imaging system according to some embodiments of the present disclosure;

FIG. 6 is a flowchart illustrating an exemplary process for correcting a respiratory amplitude of a respiratory motion of a subject according to some embodiments of the present disclosure;

FIG. 7 is a schematic diagram illustrating an exemplary surface profile of a target region of the subject according to some embodiments of the present disclosure;

FIG. 8 is a schematic diagram illustrating an exemplary installation angle and an exemplary included angle according to some embodiments of the present disclosure;

FIG. 9 is a schematic diagram illustrating an exemplary medical imaging system 900 according to some embodiments of the present disclosure;

FIG. 10 is a schematic diagram illustrating an exemplary projection component according to some embodiments of the present disclosure;

FIG. 11A and FIG. 11B are schematic diagrams illustrating the display device 10 and the medical imaging device 30 of the medical imaging system 900 in FIG. 9 according to some embodiments of the present disclosure;

FIG. 12 is a flowchart illustrating an exemplary process for helping a subject to maintain a preset status during a medical scan of the subject according to some embodiments of the present disclosure;

FIG. 13 is a flowchart illustrating an exemplary process for a foreign matter detection on a subject before a medical scan of the subject according to some embodiments of the present disclosure;

FIG. 14 is a schematic diagram illustrating an exemplary scout image of a patient according to some embodiments of the present disclosure; and

FIG. 15 is a schematic diagram illustrating an exemplary foreign matter detection image according to some embodiments of the present disclosure.

### DETAILED DESCRIPTION

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

The terminology used herein is for the purpose of describing particular example embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including,” when used in this specification, specify the presence of stated features, integers, steps, operations, elements, and/or components, but do not preclude the presence or addition of one or more other features, integers, steps, operations, elements, components, and/or groups thereof.

It will be understood that the term “system,” “engine,” “unit,” “module,” and/or “block” used herein are one method to distinguish different components, elements, parts, sections or assembly of different levels in ascending order. However, the terms may be displaced by another expression if they achieve the same purpose.

Generally, the word “module,” “unit,” or “block,” as used herein, refers to logic embodied in hardware or firmware, or to a collection of software instructions. A module, a unit, or a block described herein may be implemented as software and/or hardware and may be stored in any type of non-transitory computer-readable medium or another storage device. In some embodiments, a software module/unit/block may be compiled and linked into an executable program. It will be appreciated that software modules can be callable from other modules/units/blocks or from themselves, and/or may be invoked in response to detected events or interrupts. Software modules/units/blocks configured for execution on computing devices may be provided on a computer-readable medium, such as a compact disc, a digital video disc, a flash drive, a magnetic disc, or any other tangible medium, or as a digital download (and can be originally stored in a compressed or installable format that needs installation, decompression, or decryption prior to execution). Such software code may be stored, partially or fully, on a storage device of the executing computing device, for execution by the computing device. Software instructions may be embedded in firmware, such as an EPROM. It will be further appreciated that hardware modules/units/blocks may be included in connected logic components, such as gates and flip-flops, and/or can be included of programmable units, such as programmable gate arrays or processors. The modules/units/blocks or computing device functionality described herein may be implemented as software modules/units/blocks, but may be represented in hardware or firmware. In general, the modules/units/blocks described herein refer to logical modules/units/blocks that may be combined with other modules/units/blocks or divided into sub-modules/sub-units/sub-blocks despite their physical organization or storage. The description may be applicable to a system, an engine, or a portion thereof.

It will be understood that when a unit, engine, module, or block is referred to as being “on,” “connected to,” or “coupled to,” another unit, engine, module, or block, it may be directly on, connected or coupled to, or communicate with the other unit, engine, module, or block, or an intervening unit, engine, module, or block may be present, unless the context clearly indicates otherwise. As used herein, the term “and/or” includes any and all combinations of one or more of the associated listed items. The term “pixel” and “voxel” in the present disclosure are used interchangeably to refer to an element of an image. An anatomical structure shown in an image of a subject (e.g., a patient) may correspond to an actual anatomical structure existing in or on the subject's body.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

Respiratory motion minoring is needed in a medical scan to reduce or eliminate the effect of the respiratory motion of a scanned subject on the scanning process and on a resulting image of the medical scan. Generally, the respiratory motion monitoring is performed by using a respiratory motion detector to collect a respiratory signal of the subject. A commonly used respiratory motion detector is a radar respiratory sensor, which collects the respiratory signal by emitting detecting signals toward the subject and receiving signals reflected from the subject. Conventionally, the respiratory signal collected by the respiratory motion detector is directly used in subsequent scan data processing (e.g., for performing respiratory motion correction on the scan data).

However, in some occasions, the relative position between the body surface of the subject and the respiratory signal detector may change during the medical scan. For example, the subject may be moved to different bed positions so that different portions of the subject may be scanned, and/or the body surface of the chest of the subject may fluctuate due to the respiratory motion. The change in the relative position may result in an inconsistency between respiratory signals collected by the respiratory motion detector during the medical scan. For example, even if the subject has a uniform respiratory motion during the scan, a first respiratory signal collected by the respiratory motion detector when the subject locates at a first bed position may be different from a second respiratory signal collected by the respiratory motion detector when the subject locates at a second bed position. Hence, conventional respiratory motion monitoring approaches have a limited accuracy because of the inconsistency between respiratory signals.

To address the problems of the conventional respiratory motion monitoring approaches, the present disclosure provides systems and methods for correcting a respiratory amplitude of a respiratory motion of a subject during a medical scan. For example, the systems may determine the respiratory amplitude of the respiratory motion of the subject at a specific time point during the medical scan based on the respiratory signal relating to the respiratory motion. The respiratory signal may be collected using a respiratory motion detector by emitting detecting signals toward a target region of the subject. The systems may also obtain surface information of the target region corresponding to the time point. The systems may further correct the respiratory amplitude based on the surface information of the target region. In some embodiments, the corrected respiratory amplitude may reflect the intensity of the respiratory motion of the subject along a standard direction (e.g., a normal direction of the target region).

In some embodiments, respiratory amplitudes of the subject at multiple time points during the medical scan may be corrected. The corrected respiratory amplitudes corresponding to different time points may reflect the intensities of the respiratory motion along the standard direction. In such cases, the corrected respiratory amplitudes may be comparable and accurate, and the effect of the change in the relative position between the respiratory motion detector and the body surface of the subject may be reduced or eliminated.

Compared with the conventional respiratory motion monitoring approaches, the systems of the present disclosure may obtain more accurate respiratory amplitude of the subject by correcting the respiratory amplitude based on the surface information of the target region of the subject. Hence, the subsequent scan data processing based on the corrected respiratory amplitude of the subject may be improved, thereby improving the imaging quality of the medical scan by reducing or eliminating, for example, respiratory motion-induced artifacts in a resulting image.

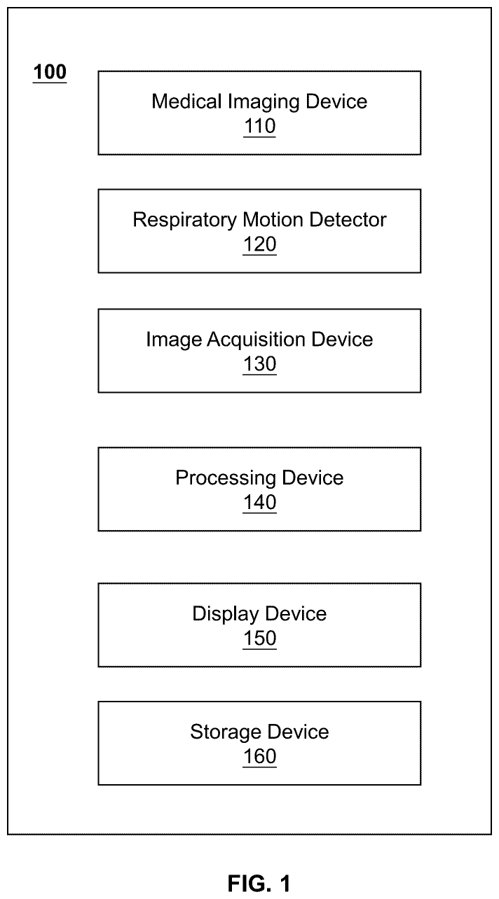

FIG. 1 is a schematic diagram illustrating an exemplary medical imaging system 100 according to some embodiments of the present disclosure.

As shown in FIG. 1, the medical imaging system 100 may include a medical imaging device 110, a respiratory motion detector 120, an image acquisition device 130, a processing device 140, a display device 150, and a storage device 160. In some embodiments, the components of the medical imaging system 100 may be connected to and/or communicate with each other via a wireless connection, a wired connection, or a combination thereof. The connections between the components in the medical imaging system 100 may be variable. For example, the medical imaging device 110 may be connected to the processing device 140 through a network. As another example, the medical imaging device 110 may be connected to the processing device 140 directly.

The medical imaging device 110 may be configured to scan a subject (or a part of the subject) to acquire medical image data associated with the subject. The medial image data relating to the patient may be used for generating an anatomical image of the subject. The anatomical image may illustrate an internal structure of the subject. The subject may be biological or non-biological. For example, the subject may include a patient, a man-made object, etc. As another example, the subject may include a specific portion, an organ, and/or tissue of the patient. Specifically, the subject may include the head, the neck, the thorax, the heart, the stomach, a blood vessel, soft tissue, a tumor, or the like, or any combination thereof. In the present disclosure, “object” and “subject” are used interchangeably.

In some embodiments, the medical imaging device 110 may include a single modality imaging device. For example, the medical imaging device 110 may include a positron emission tomography (PET) device, a single-photon emission computed tomography (SPECT) device, a magnetic resonance imaging (MRI) device (also referred to as an MR device, an MR scanner), a computed tomography (CT) device, an ultrasound (US) device, an X-ray imaging device, or the like, or any combination thereof. In some embodiments, the medical imaging device 110 may include a multi-modality imaging device. Exemplary multi-modality imaging devices may include a PET-CT device, a PET-MRI device, a SPET-CT device, or the like, or any combination thereof. The multi-modality imaging device may perform multi-modality imaging simultaneously or in sequence. For example, the PET-CT device may generate structural X-ray CT data and functional PET data simultaneously in a single scan or in sequence in multiple scans. The PET-MRI device may generate MRI data and PET data simultaneously in a single scan or in sequence in multiple scans.

The respiratory motion detector 120 may be configured to collect a respiratory signal that reflects the respiratory motion of the subject. For example, the respiratory motion detector 120 may collect a respiratory signal of a respiratory motion of the subject during a medical scan of the subject performed by the medical imaging device 110. In some embodiments, the respiratory motion detector 120 may be a device with distance sensing ability, which can obtain fluctuation data relating to the fluctuation of the body surface of the subject caused by the subject's the respiratory motion. In some embodiments, the respiratory motion detector 120 may collect the respiratory signal by emitting detecting signals toward the subject. Specifically, the respiratory motion detector 120 may emit the detecting signals to toward the subject, and the detecting signals may be reflected by the subject. The respiratory motion detector 120 may receive at least a portion of the reflected signals (e.g., an echo signal). The respiratory signal may be generated based on the received reflected signals. For example, a signal with a certain periodicity may be extracted from the reflected signals, and designated as the respiratory signal. Exemplary respiratory motion detector 120 may include an ultrasonic detector, an infrared detector, a radar sensor, or the like, or any combination thereof. The ultrasonic detector may emit ultrasonic waves toward the subject, which has a strong penetrability and a low cost. The infrared detector operates by sensing heat signals, which has a high reliability and a low power consumption. The radar sensor may emit radar signals toward the subject.

In some embodiments, the radar sensor may include a millimeter wave radar sensor, which has a small size, a small weight, and a strong anti-interference ability. The millimeter wave radar sensor may transmit radar signals with a wavelength which is in a millimeter (mm) range (e.g., 1˜10 mm). In some embodiments, the emission frequency of the millimeter wave radar sensor may be in a range of 30˜300 GHz. A high frequency range (e.g., 30˜300 GHz) of the millimeter wave radar sensor may be used to detect a body surface movement (e.g., a skin movement) of the subject. In some embodiments, the radar sensor may include a modulated continuous wave radar (e.g., a frequency modulated continuous wave (FMCW) radar), an unmodulated continuous-wave radar, or the like.

In some embodiments, the respiratory motion detector 120 may be mounted at any suitable location for monitoring the respiratory motion of the subject. In some embodiments, the respiratory motion detector 120 may be integrated into or mounted on the medical imaging device 110. In some embodiments, the respiratory motion detector 120 may be mounted outside a field of view (FOV) of the medical imaging device 110 (e.g., on a main magnet of an MRI device), in order to reduce or eliminate the signal interference between the respiratory motion detector 120 and the medical imaging device 110 (e.g., the MRI device).

In some embodiments, the mounting location of the respiratory motion detector 120 may be determined based on the FOV of the respiratory motion detector 120, and the FOV of the medical imaging device 110. For example, the respiratory motion detector 120 may be mounted at a specific location such that the FOV of the respiratory motion detector 120 can cover at least part of the FOV of the medical imaging device 110. In some embodiments, a plurality of respiratory motion detectors 120 may be mounted at different positions. The number (or count) of the respiratory motion detectors 120 may be determined based on the FOV of each of the respiratory motion detectors 120, the FOV of the medical imaging device 110, and/or a mounting location of each of the respiratory motion detectors 120. For example, each of the plurality of respiratory motion detectors 120 may be mounted at a specific location such that a total FOV of the plurality of respiratory motion detectors 120 can cover the FOV of the medical imaging device 110.

The image acquisition device 130 may be configured to capture an optical image of the subject, which may illustrate an external body surface of the subject. For example, the image acquisition device 130 may be configured to capture one or more optical images of the subject during the medical scan of the subject performed by the medical imaging device 110. The image acquisition device 130 may be and/or include any suitable device capable of capturing optical images of subjects located in a field of view of the image acquisition device 130. For example, the image acquisition device 130 may include a camera (e.g., a digital camera, an analog camera, a binocular camera, etc.), a red-green-blue (RGB) sensor, an RGB-depth (RGB-D) sensor, a time-of-flight (TOF) camera, a depth camera, a structure light camera, a laser radar, or the like, or any combination thereof.

The laser radar may emit signals (i.e., laser beams) toward the subject, and compare echo signals reflected from the subject with the emitted signals to obtain information relating to the subject, such as a position, an azimuth, an altitude, or the like, or any combination thereof, of the subject. The binocular camera uses two cameras arranged at different positions to obtain images of the object, and obtains coordinates of the subject in coordinate systems of the two cameras, respectively. As long as the two cameras are calibrated in advance, the coordinates of the subject in the coordinate system of one of the two cameras may be obtained based on a geometric position relationship between the two cameras, that is, the position of the subject may be determined. The structure light camera may determine information, such as position information and depth information, of the subject according to a change of light signals projected to the subject, and then generate a 3D model of the subject based on the determined information. The TOF camera may continuously emit light pulses to the subject, and then receive light pulses reflected from the subject using one or more sensors. Depth information of the subject may be obtained by detecting the TOF of these emitted and received light pulses. In some embodiments, the optical image(s) captured by the image acquisition device 130 may include three-dimensional (3D) surface information of the subject, such as depth information, point cloud information, TOF information, or the like, or any combination thereof.

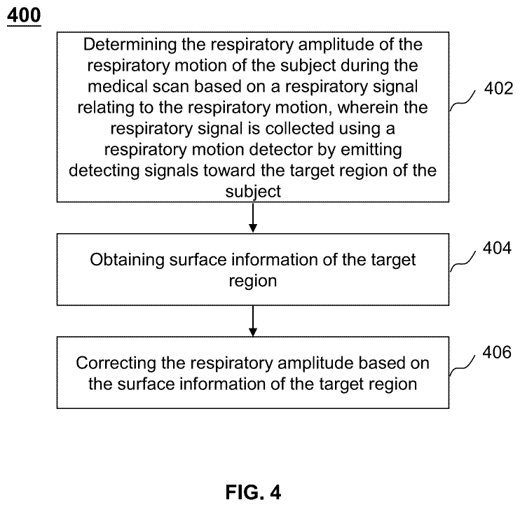

In some embodiments, the image acquisition device 130 may be mounted at any suitable location for acquiring optical images of the subject. In some embodiments, the determination of the mounting location of the image acquisition device 130 may be performed in a similar manner as that of the mounting location of the respiratory motion detector 120. In some embodiments, a plurality of image acquisition devices 130 may be mounted at different positions. In some embodiments, the image acquisition device 130 and the respiratory motion detector 120 may be mounted on a same side or different sides of a scanning tunnel of the medical imaging device 110. More descriptions for the respiratory motion detector 120 and the image acquisition device 130 may be found elsewhere in the present disclosure (e.g., FIG. 4 and the descriptions thereof).

The display device 150 may be configured to display information (e.g., images, videos, etc.) received from other components (e.g., the image acquisition device 130, the processing device 140, the storage device 160) of the medical imaging system 100. For example, the display device 140 may include a projector disposed in the scanning tunnel of the medical imaging device 110. The projector may be configured to project image data (e.g., an image, a video, a virtual character) on an inside wall of the scanning tunnel. As another example, the display device 140 may include a liquid crystal display (LCD), a light emitting diode (LED)-based display, a flat panel display or curved screen (or television), a cathode ray tube (CRT), or the like, or a combination thereof. In some embodiments, the display device 140 may be an immersive display device, such as, a virtual reality device, an augmented reality device, a mixed reality device, etc., worn by the subject. For example, the immersive display may be a head-mounted display. The head-mounted display may include a set of glasses or goggles covering the subject's eyes.

In some embodiments, during a medical scan of the subject performed by the medical imaging device 110, the display device 150 may display preset image data to attract the subject's attention, so that the subject can maintain a preset status (e.g., a status without pose motion, a preset physiological motion status, etc.). In some embodiments, the display content and/or the display manner of the display device 150 may be determined according to the status of the subject during the medical scan of the subject. For example, the display device 150 may display a preset video to the subject in a preset status, then when the status of the subject changes from the preset status to another status, the display device 150 may switch the preset video to an image to remind the subject to maintain the preset status. More descriptions for the display device 150 may be found elsewhere in the present disclosure (e.g., FIGS. 9-12 and the descriptions thereof).

The processing device 140 may be configured to process data and/or information obtained from one or more components (e.g., the medical imaging device 110, the respiratory motion detector 120, the image acquisition device 130, the storage device 160, etc.) of the medical imaging system 100. For example, during a medical scan of the subject performed by the medical imaging device 110, the processing device 140 may determine a respiratory amplitude of the subject based on a respiratory signal of the subject detected by the respiratory motion detector 120. The processing device 140 may also determine surface information of a target region (e.g., the chest) of the subject based on an optical image of the subject captured by the image acquisition device 130. Further, the processing device 140 may correct the respiratory amplitude based on the surface information of the target region. As another example, during the medical scan of the subject, the processing device 140 may determine the display content and/or the display manner of the display device 150 based on the motion (e.g., the respiratory motion, the rigid body motion) of the subject. As still another example, before the medical scan of the subject, the processing device 140 may obtain a scout image of the subject collected by a scout scan, and perform a foreign matter detection on the scout image. Further, the processing device 140 may determine whether the medical scan can be started based on a result of the foreign matter detection (also referred to as a foreign matter detection result). Foreign matter disposed on or within the subject may include one or more objects that are not naturally produced or grow by the subject but is on or inside the subject.

In some embodiments, the processing device 140 may be a single server or a server group. The server group may be centralized or distributed. In some embodiments, the processing device 140 may be local or remote. Merely for illustration, only one processing device 140 is described in the medical imaging system 100. However, it should be noted that the medical imaging system 100 in the present disclosure may also include multiple processing devices. Thus operations and/or method steps that are performed by one processing device 140 as described in the present disclosure may also be jointly or separately performed by the multiple processing devices. For example, if in the present disclosure the processing device 140 of the medical imaging system 100 executes both process A and process B, it should be understood that the process A and the process B may also be performed by two or more different processing devices jointly or separately in the medical imaging system 100 (e.g., a first processing device executes process A and a second processing device executes process B, or the first and second processing devices jointly execute processes A and B).

The storage device 160 may be configured to store data, instructions, and/or any other information. For example, the storage device 160 may store data obtained from the medical imaging device 110, the respiratory motion detector 120, the image acquisition device 130, and the processing device 140. For example, the storage device 160 may store an optical image captured by the image acquisition device 130, a respiratory signal collected by the respiratory motion detector 120, etc. In some embodiments, the storage device 160 may store data and/or instructions that the processing device 140 may execute or use to perform exemplary methods described in the present disclosure.

This description is intended to be illustrative, and not to limit the scope of the present disclosure. Many alternatives, modifications, and variations will be apparent to those skilled in the art. The features, structures, methods, and characteristics of the exemplary embodiments described herein may be combined in various ways to obtain additional and/or alternative exemplary embodiments. However, those variations and modifications do not depart the scope of the present disclosure. Merely by way of example, the medical imaging system 110 may include one or more additional components and/or one or more components described above may be omitted. For example, the medical imaging system 110 may include a network. The network may include any suitable network that can facilitate the exchange of information and/or data for the medical imaging system 110. In some embodiments, one or more components of the medical imaging system 110 (e.g., the medical imaging device 110, the respiratory motion detector 120, the image acquisition device 130, the processing device 140, the display device 150, the storage device 160, etc.) may communicate information and/or data with one or more other components of the medical imaging system 110 via the network.

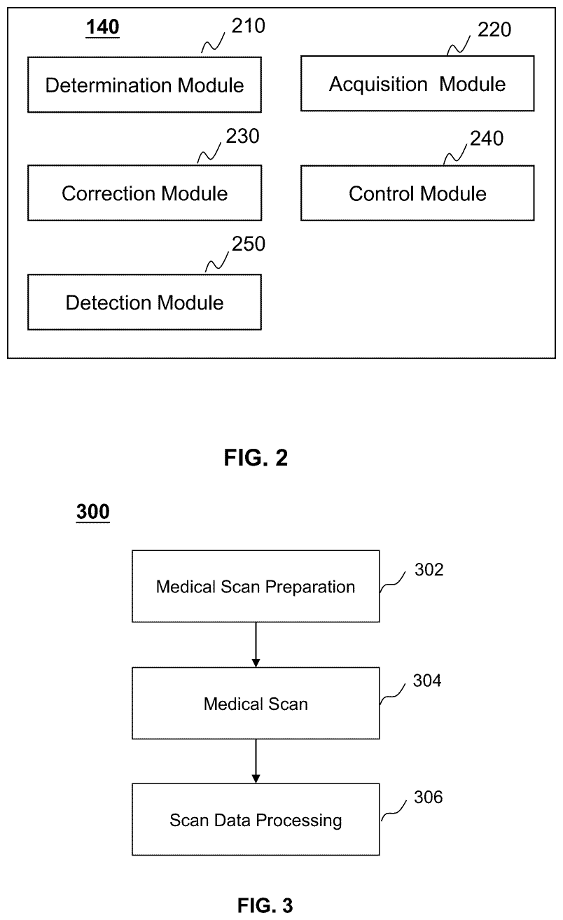

FIG. 2 is a block diagram illustrating exemplary processing device 140 according to some embodiments of the present disclosure.

As shown in FIG. 2, the processing device 140 may include a determination module 210, an acquisition module 220, a correction module 230, a control module 240, and a detection module 250.

The determination module 210 may be configured to determine a respiratory amplitude of the respiratory motion of the subject during the medical scan based on a respiratory signal relating to the respiratory motion. The respiratory amplitude may reflect the intensity of the respiratory motion of the subject at a first time point. More descriptions regarding the determining of the respiratory amplitude of the respiratory motion of the subject during the medical scan based on the respiratory signal relating to the respiratory motion may be found elsewhere in the present disclosure. See, e.g., operation 402 in FIG. 4, and relevant descriptions thereof.

In some embodiments, the determination module 210 may be configured to determine motion data of the subject during the medical scan of the subject based on at least one of respiratory motion data or posture data, and whether the subject has an obvious motion at a third time point during the medical scan based on the motion data of the subject. More descriptions regarding the determining of the motion data of the subject and whether the subject has an obvious motion at the third time point during the medical scan based on motion data of the subject may be found elsewhere in the present disclosure. See, e.g., operations 1202 and 1204 in FIG. 12, and relevant descriptions thereof.

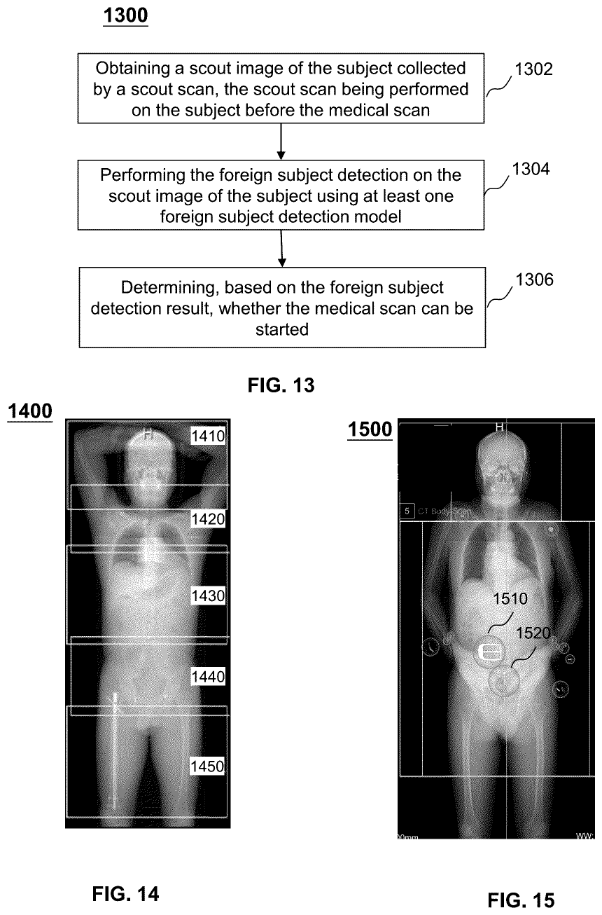

In some embodiments, the determination module 210 may be configured to determine whether the medical scan can be started based on the foreign matter detection result. More descriptions regarding the determining of whether the medical scan can be started based on the foreign matter detection result may be found elsewhere in the present disclosure. See, e.g., operation 1306 in FIG. 13, and relevant descriptions thereof.

The acquisition module 220 may be configured to obtain information relating to the medical imaging system 100. For example, the acquisition module 220 may be configured to obtain surface information of the target region. More descriptions regarding the obtaining of the surface information of the target region may be found elsewhere in the present disclosure. See, e.g., operation 404 in FIG. 4, and relevant descriptions thereof. As another example, the acquisition module 220 may be configured to obtain a scout image of the subject collected by a scout scan. The scout scan may be performed on the subject before the medical scan. More descriptions regarding the obtaining of the scout image of the subject collected by the scout scan may be found elsewhere in the present disclosure. See, e.g., operation 1302 in FIG. 13, and relevant descriptions thereof.

The correction module 230 may be configured to correct the respiratory amplitude based on the surface information of the target region. In some embodiments, the corrected respiratory amplitude may reflect the intensity of the respiratory motion of the subject along a standard direction (e.g., a normal direction of the target region). More descriptions regarding the correcting of the respiratory amplitude based on the surface information of the target region may be found elsewhere in the present disclosure. See, e.g., operation 406 in FIG. 4, and relevant descriptions thereof.

The control module 240 may be configured to control a display device to perform a target operation. In some embodiments, the target operation may include stopping displaying, changing the display content, etc. More descriptions regarding the controlling of the display device to perform the target operation may be found elsewhere in the present disclosure. See, e.g., operation 1204 in FIG. 4, and relevant descriptions thereof.

The detection module 250 may be configured to perform the foreign matter detection on the scout image of the subject using at least one foreign matter detection model. More descriptions regarding the performing of the foreign matter detection on the scout image of the subject using at least one foreign matter detection model may be found elsewhere in the present disclosure. See, e.g., operation 1304 in FIG. 13, and relevant descriptions thereof.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, the processing device 140 may include one or more additional modules, such as a storage module (not shown) for storing data.

FIG. 3 is a schematic diagram illustrating an exemplary medical imaging process 300 according to some embodiments of the present disclosure.

In 302, medical scan preparation may be performed.

The medical scan preparation may include one or more preparation operations including, for example, positioning a subject to be scanned to be in a suitable scanning position, selecting a scanning protocol, debugging a medical imaging device, performing a foreign matter detection on the subject, etc. Merely by way of example, the foreign matter detection may be performed on the subject to ensure the safety and the quality of the subsequent medical scan.

In some embodiments, the foreign matter detection may be performed manually by a user. For example, the user may inquiry the subject whether he/she carries foreign matter, or visually inspect and/or manually check whether the subject carries foreign matter. In some embodiments, in order to improve the accuracy and efficiency of the foreign matter detection, the processing device 140 may perform the foreign matter detection automatically without user intervention or with limited user intervention. Specifically, the processing device 140 may obtain a scout image of the subject collected by a scout scan. The processing device 140 may perform a foreign matter detection on the scout image of the subject using at least one foreign matter detection model to generate a foreign matter detection result. Further, the processing device 140 may determine whether the medical scan can be started based on the foreign matter detection result. More descriptions for the foreign matter detection performed on the subject may be found elsewhere in the present disclosure (e.g., FIG. 13 and the descriptions thereof).

In response to determining that the medical scan can be started, the processing device 140 may directly send an instruction to the medical imaging device to direct the medical imaging device to perform the medical scan. Alternatively, the processing device 140 may generate a prompt information indicating that the medical scan can be started, and send the prompt information to a user. In response to determining that the medical scan can not be started, the processing device 140 may generate a prompt information indicating why the medical scan can not be started, and/or corresponding suggestions. For example, if it is determined that non-iatrogenic foreign matter is disposed on or within the subject, the processing device 140 may generate first prompt information for requiring the subject to take off the one or more non-iatrogenic foreign matters.

In 304, the medical scan may be performed on the subject via the medical imaging device (e.g., the medical imaging device 110).

In some embodiments, the subject (e.g., a patient) may move (e.g., have a rigid body motion and/or a physiological motion) during the medical scan. The motion of the subject during the medical scan may affect imaging quality (e.g., cause motion artifacts in a resulting image), which may hinder an accurate detection, localization, and/or quantification of possible lesions (e.g., a tumor). Therefore, in some embodiments, a display device (e.g., the display device 150) may be configured to display image data to the subject during the medical scan. If the subject has an obvious motion, the display content and/or display manner of the display device may be changed to attract the subject's attention and keep the subject still. For example, the processing device 140 may determine whether the subject has an obvious motion during the medical scan based on motion data of the subject. In response to determining that the subject has an obvious motion, the processing device 140 may control the display device to perform a target operation to attract the subject's attention to remind the subject to remain a preset status. More descriptions for the helping a subject to maintain a preset status during a medical scan of the subject may be found elsewhere in the present disclosure (e.g., FIGS. 9-12 and the descriptions thereof).

In some embodiments, a scanned region of the subject may be located within an FOV of the medical imaging device, and the scanned region may be scanned during the medical scan. If the scanned region of the subject includes a region (e.g., the abdomen, the chest, etc.) that is influenced by a respiratory motion of the subject, the respiratory motion of the subject may affect the quality of an image of the subject generated based on scan data obtained by the medical scan. For brevity, a region that is influenced by a respiratory motion of the subject is referred to as a respiratory region herein. Moreover, as described elsewhere in this disclosure, the relative position between the body surface of the subject and the respiratory signal detector may change during the medical scan, thereby resulting in an inconsistency between respiratory signals collected by the respiratory motion detector during the medical scan. Therefore, data relating to the respiratory motion of the subject needs to be collected and corrected, and the scan data of the subject may be processed based on the corrected data relating to the respiratory motion, so that the image of the subject obtained based on the processed scan data may have an improved accuracy.

In some embodiments, the processing device 140 may obtain surface information of the scanned region (e.g., based on an 3D optical image of the subject captured by an image acquisition device) before or during the medical scan. The processing device 140 may further determine whether the scanned region include a respiratory region of the subject based on the surface information of the scanned region. Alternatively, information indicating whether the scanned region include a respiratory region may be manually inputted into the medical imaging system 100 by a user. In response to determining that the scanned region include a respiratory region, the respiratory motion detector may be direct to collect a respiratory signal relating to the respiratory motion of the subject during the medical scan. The respiratory signal may be sent to the processing device 140 for analysis. Merely by way of example, the processing device 140 may perform process 400 as described in connection with FIG. 4 to correct a respiratory amplitude of the respiratory motion of the subject during the medical scan.

In 306, after the medical scan is completed, the scan data collected by the medical scan may be processed. In some embodiments, one or more medical images of the subject may be generated based on the processed scan data. For example, a plurality of medical images corresponding to a plurality of respiratory phases may be generated. As another example, a combined image may be generated by combining the medical images. More descriptions for the processing of the scan data may be found elsewhere in the present disclosure (e.g., operation 406 in FIG. 4 and the descriptions thereof).

FIG. 4 is a flowchart illustrating an exemplary process 400 for correcting a respiratory amplitude of a respiratory motion of a subject during a medical scan according to some embodiments of the present disclosure. In some embodiments, the process 400 may be implemented in the medical imaging system 100 illustrated in FIG. 1. For example, the process 400 may be stored in the storage device 160 of the medical imaging system 100 as a form of instructions, and invoked and/or executed by the processing device 140 (e.g., one or more modules as illustrated in FIG. 2). The operations of the illustrated process presented below are intended to be illustrative. In some embodiments, the process 400 may be accomplished with one or more additional operations not described, and/or without one or more of the operations discussed. Additionally, the order in which the operations of the process 400 as illustrated in FIG. 4 and described below is not intended to be limiting.

In 402, the processing device 140 (e.g., the determination module 210) may determine a respiratory amplitude of the respiratory motion of the subject during the medical scan based on a respiratory signal relating to the respiratory motion.

The respiratory amplitude may reflect the intensity of the respiratory motion of the subject at a first time point. In some embodiments, the respiratory signal may be collected using a respiratory motion detector by emitting detecting signals toward a target region of the subject. The target region of the subject refers to a region of the subject that is within an FOV of the respiratory motion detector at the first time point. In some embodiments, the target region may include at least a portion of a scanned region of the subject that needs to receive the medical scan at the first time point. In some embodiments, the target region may include a respiratory region (e.g., the abdomen, the chest, etc.) of the subject that is influenced by the respiratory motion. The respiratory motion detector may be any suitable respiratory sensor (e.g., the respiratory motion detector 120) having respiratory motion detection functions.

The respiratory signal may reflect the motion of tissue or an organ that is caused or influenced by the respiratory motion of the subject. In some embodiments, the respiratory signal may include information relating to the respiratory motion of the subject. The information relating to the respiratory motion may include a respiratory cycle, a respiratory amplitude (or displacement), a respiratory rate, and/or a respiratory frequency, or the like, or any combination thereof, of the subject. The respiratory cycle may include a plurality of respiratory phases, such as an inspiratory phase (during which the chest of the subject expands and air flows into the lungs) and an expiratory phase (during which the chest shrinks and air is pushed out of the lungs). The processing device 140 may determine the respiratory amplitude based on the information relating to the respiratory motion. For example, the respiratory signal may be represented as a respiratory amplitude curve reflecting a change of respiratory amplitude with time. The processing device 140 may determine the respiratory amplitude at the specific time according to the respiratory amplitude curve.

In some embodiments, the processing device 140 may directly obtain the respiratory signal from the respiratory motion detector. Alternatively, the respiratory signal may be collected by the respiratory motion detector and stored in a storage device (e.g., the storage device 160, or an external source). The processing device 140 may retrieve the respiratory signal from the storage device.

In 404, the processing device 140 (e.g., the acquisition module 220) may obtain surface information of the target region.

The surface information of the target region may reflect the contour of the body surface of target region at the first time point. For example, the surface information of the target region may include shape information, size information, position information, or the like, or any combination thereof, of the body surface of the target region. Merely by way of example, the surface information may include a distance between each physical point on the body surface of the target region and a reference object (e.g., the scanning table).

In some embodiments, a 3D optical image of the subject may be acquired using an image acquisition device (e.g., the image acquisition device 130). The 3D optical image may be generated, stored, or presented in a form of an image, a video frame, etc. For example, if the medical scanning process takes a relatively short time, the 3D optical image may be a 3D image; if the scanning process takes a relatively long time, the 3D optical image may be a video frame. The 3D optical image may include, for example, a 3D point cloud image, a depth image (or range image), etc. In some embodiments, the processing device 140 may determine an initial 3D optical image captured by the image acquisition device. The processing device 140 may determine the 3D optical image based on the initial 3D optical image. In some embodiments, the 3D optical image may be a 3D optical image looking at the subject from a position of the respiratory motion detector (e.g., a 3D optical image captured at the position of the respiratory motion detector using the image acquisition device). In some embodiments, the processing device 140 may obtain position information of the image acquisition device and position information of the respiratory motion detector. The position information of the image acquisition device (or the respiratory motion detector) may include an installation angle, position coordinates, etc., of the image acquisition device (or the respiratory motion detector). The processing device 140 may further transform the initial 3D optical image according to the position information of the image acquisition device and the position information of the respiratory motion detector to obtain the 3D optical image.

The processing device 140 may determine the surface information of the target region based on the 3D optical image of the subject. For example, the processing device 140 may determine the shape information, the size information, the position information, or the like, of the body surface of the target region based on the 3D optical image of the subject. In some embodiments, a target 3D optical image of the target region may be segmented from the 3D optical image of the subject, and the target 3D optical image of the target region may be designated as the surface information of the target region. For example, a portion representing the chest of the subject may be segmented from a depth image of the subject, and the segmented portion may include depth information of each physical point on the chest of the subject and be designated as the surface information of the chest.

In some embodiments, the 3D optical image may be captured by the image acquisition device 130 at the first time point or a specific time point close to the first time point (e.g., an interval between the first and specific time points is shorter than a threshold), and the 3D optical image and the surface information determined based on such 3D optical image may be deemed as being corresponding to the first time point.

In some embodiments, the image acquisition device and the respiratory motion detector may be mounted on a same side or different sides of a scanning tunnel of the medical imaging device. In some embodiments, installation angles of the respiratory motion detector and the image acquisition device may be the same or different. For example, an installation angle of a device may be represented by an angle between a surface of the device and a reference plane (e.g., a plane parallel to the scanning table where the subject lies on). In some embodiments, at least one of the installation angles of the respiratory motion detector and the image acquisition device may be adjustable. For example, the respiratory motion detector may be installed in a hinged manner and its installation angle may be adjustable. Before the medical scan, the installation angle of the respiratory motion detector may be adjusted to a suitable value to cover the scanned region as much as possible.

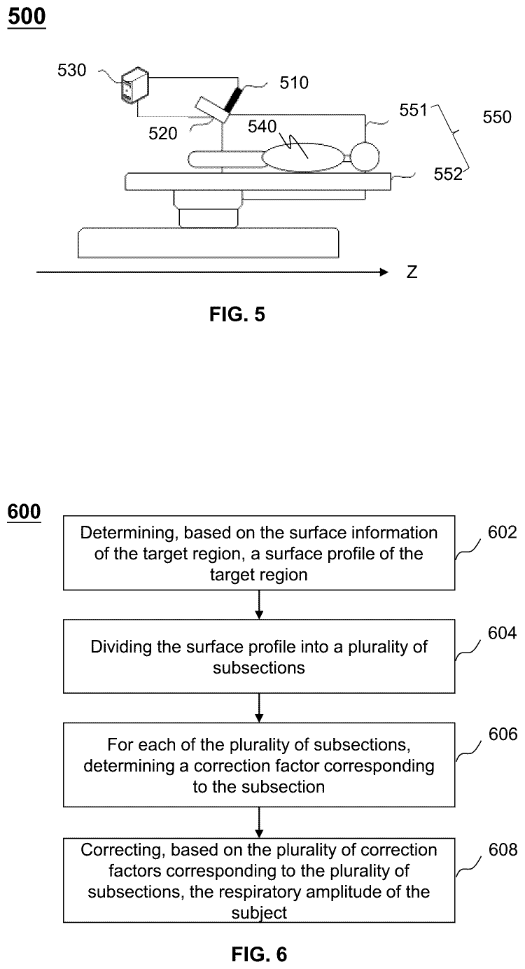

Merely by way of example, FIG. 5 is a schematic diagram illustrating an exemplary installation positions of a respiratory motion detector and an image acquisition device in a medical imaging system 500 according to some embodiments of the present disclosure. In some embodiments, the medical imaging system 500 may be an exemplary embodiment of the medical imaging system 100 as described in FIG. 1. As shown in FIG. 5, the medical imaging system 500 may include a respiratory motion detector 510, an image acquisition device 520, a processing device 530, and a medical imaging device 550. The medical imaging device 550 may include a scanning tunnel 551 and a scanning table 552. The scanning table 552 may move a subject to be scanned into the scanning tunnel 551 along a longitudinal direction (i.e., a Z-direction in FIG. 5) of the scanning tunnel 551 for receiving a medical scan. The respiratory motion detector 510 and the image acquisition device 520 may be mounted on a same side of the scanning tunnel 511. An installation angle of the respiratory motion detector 510 may be represented by an angle between a surface of the respiratory motion detector 510 for emitting signals and a plane parallel to the scanning table 552. An installation angle of the image acquisition device 520 may be represented by an angle between a surface of the image acquisition device 520 for shooting the subject and a plane parallel to the scanning table 552. In some embodiments, the installation angles of the respiratory motion detector 510 and the image acquisition device 520 may be the same.

In 406, the processing device 140 (e.g., the correction module 230) may correct the respiratory amplitude based on the surface information of the target region.

In some embodiments, the corrected respiratory amplitude may reflect the intensity of the respiratory motion of the subject along a standard direction (e.g., a normal direction of the target region). In some embodiments, the processing device 140 may determine a surface profile (or contour) of the target region based on the surface information of the target region. The processing device 140 may correct the respiratory amplitude of the subject based on the surface profile. More descriptions for the correction of the respiratory amplitude of the subject based on the surface profile may be found elsewhere in the present disclosure (e.g., FIG. 6 and the descriptions thereof).

In some embodiments, the processing device 140 may determine a plurality of respiratory amplitudes of the respiratory motion at a plurality of time points during the medical scan based on the respiratory signal relating to the respiratory motion. The processing device 140 may obtain sets of surface information of the target region of the subject. Each of the sets of surface information may correspond to one of the plurality of time points. For each of the plurality of time points, the processing device 140 may correct the respiratory amplitude at the time point based on the surface information corresponding to the time point. In such cases, multiple corrected respiratory amplitudes corresponding to the multiple time points may be obtained. The corrected respiratory amplitudes corresponding to different time points may reflect the intensities of the respiratory motion along the standard direction. In some embodiments, the determination of a respiratory amplitude and the correction of the respiratory amplitude may be performed continuously or intermittently (e.g., periodically) during the medical scan. In some embodiments, the subject may be moved to different bed positions so that different portions of the subject may be scanned. When the subject is moved to a specific bed position, one or more corrected respiratory amplitudes corresponding to the specific bed position may be obtained.

In some embodiments, the processing device 140 may obtain scan data of the subject collected by medical scan. Further, the processing device 140 may process the scan data of the subject based on the corrected respiratory amplitudes corresponding to the time points.

In some embodiments, the processing device 140 may determine a plurality of respiratory phases of the respiratory motion of the subject. The respiratory motion of the subject may include a plurality of respiratory cycles, and each respiratory cycle may include a plurality of respiratory phases. A respiratory phase may correspond to or indicate a specific respiratory state of the subject. Exemplary respiratory phases in a respiratory cycle may include an initial stage of inspiration, an end stage of inspiration, an initial stage of expiration, an end stage of expiration, etc. In some embodiments, the processing device 140 may determine a respiratory motion curve based on the multiple corrected respiratory amplitudes corresponding to the multiple time points. For example, the respiratory motion curve may be established with time as a horizontal axis and a corrected respiratory amplitude as a vertical axis. In some embodiments, the plurality of respiratory phases may be determined based on at least one portion of the respiratory motion curve of the subject. For example, an end stage of inspiration may correspond to a peak position in the respiratory motion curve. An end stage of expiration may correspond to a trough position in the respiratory motion curve.

The processing device 140 may divide the scan data into a plurality of sets of scan data each of which corresponds to one of the plurality of respiratory phases. For each of the plurality of respiratory phases, the processing device 140 may generate a reconstruction image based on the corresponding set of scan data using one or more reconstruction algorithms. Exemplary reconstruction algorithms may include a rapid reconstruction, an algebraic reconstruction, an iteration reconstruction, a back projection reconstruction, or the like, or any combination thereof. Exemplary rapid reconstruction algorithms may include fast Fourier transform, a compressed sensing algorithm, a deep learning algorithm, or the like, or any combination thereof.

In some embodiments, the processing device 140 may select a target respiratory phase from the plurality of respiratory phases. For each respiratory phase other than the target respiratory phase, the processing device 140 may transform the corresponding reconstruction image into a transformed reconstruction image corresponding to the target respiratory phase based on the corrected respiratory amplitudes of the respiratory phase and the target respiratory phase. The processing device 140 may further generate a target reconstruction image corresponding to the target respiratory phase based on the reconstruction image corresponding to the target respiratory phase and the transformed reconstruction image corresponding to each respiratory phase other than the target respiratory phase, for example, by performing image combination.

In some embodiments, the processing device 140 may determine whether the subject has an obvious rigid body motion from a second time point to the first time point. The second time point may be a time point earlier than the first time point. For example, the processing device 140 may obtain a reference 3D optical image of the subject captured by the image acquisition device at the second time point. The processing device 140 may determine a first posture (e.g., a first position and/or a first pose) of the subject at the first time point based on the 3D optical image corresponding to the first time point, and a second posture at the second time point based on the reference 3D optical image corresponding to the second time point. The processing device 140 may determine whether a change from the second posture to the first posture is greater than a first threshold. If the change from the second posture to the first posture is greater than the first threshold, it may indicate that the subject has an obvious rigid body motion from the second time point to the first time point, which may affect the quality of an image to be reconstructed based on the scan data. In some embodiments, in response to determining that the subject has an obvious rigid body motion, the processing device 140 may cause the respiratory motion detector to terminate or pause collecting the respiratory signal and/or the medical imaging device to terminate or pause acquiring the scan data of the subject. In some embodiments, in response to determining that the subject has an obvious rigid body motion, the processing device 140 may mark a portion of the respiratory signal and the scan data obtained when the subject has the rigid body motion (i.e., the corrected respiratory amplitudes and the scan data obtained during a time period between the first time point and the second time point), and the marked portion of the respiratory signal and the marked scan data may be discarded and not used for image reconstruction.

FIG. 6 is a flowchart illustrating an exemplary process 600 for correcting a respiratory amplitude of a respiratory motion of a subject according to some embodiments of the present disclosure. In some embodiments, one or more operations of the process 600 may be performed to achieve at least part of operation 406 as described in connection with FIG. 4.

In 602, the processing device 140 (e.g., the correction module 230) may determine, based on the surface information of the target region, a surface profile of the target region.

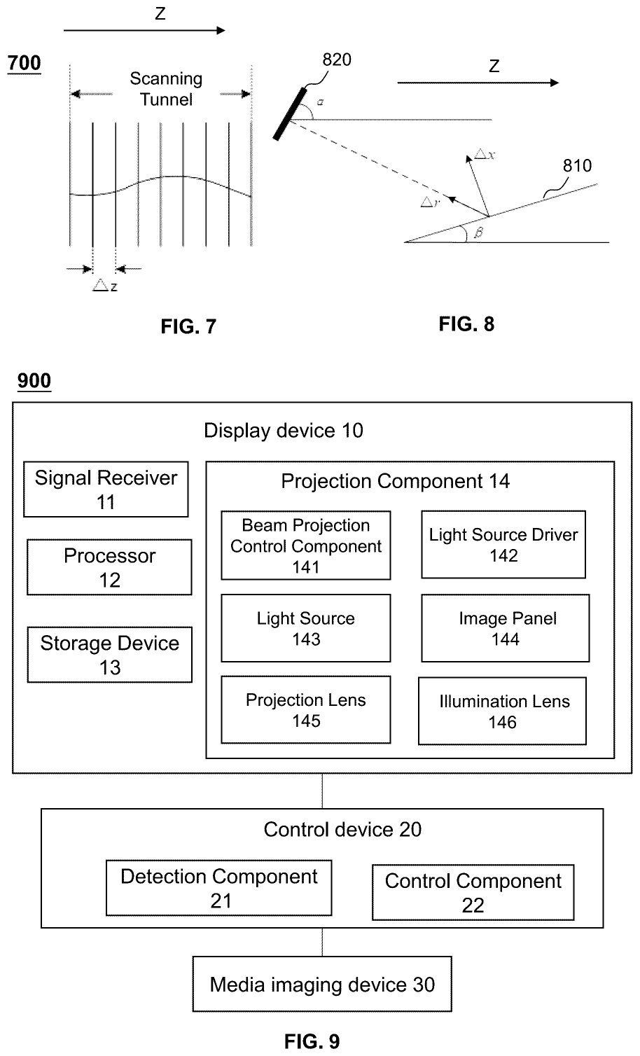

In some embodiments, the surface profile may reflect the contour of the target region. For example, the surface profile may be represented by a contour curve showing the contour of the body surface of the target region seen from a projection direction. Merely by way of example, FIG. 7 is a schematic diagram illustrating an exemplary surface profile 700 of a target region of the subject according to some embodiments of the present disclosure. As shown in FIG. 7, the surface profile 700 may be represented as a contour curve of the target region seen from a direction perpendicular to a sagittal plane of the subject, which may be generated by projecting the body surface of the target region along the direction perpendicular to the sagittal plane of the subject. A vertical coordinate of a point in the surface profile 700 may be determined based on a distance between the scanning table where the subject lies on and a physical point corresponding to the point. In some embodiments, the surface profile may be represented by a curved surface showing the body surface of the target region.

In some embodiments, the surface information of the target region may include shape information, size information, position information, or the like, of the body surface of the target region. The processing device 140 may determine the surface profile of the target region based on the shape information, the size information, the position information, or the like, of the body surface of the target region. As another example, a user or the processing device 140 may depict the surface profile of the target region in the 3D optical image of the subject or the target 3D optical image of the target region.

In 604, the processing device 140 (e.g., the correction module 230) may divide the surface profile into a plurality of subsections.

Each of the subsections may correspond to a region of the surface profile. In some embodiments, the processing device 140 may divide the surface profile into the subsections according to a preset rule. For example, the processing device 140 may evenly divide the surface profile into the subsections. For illustration purposes, the division of a contour curve is described hereinafter. In some embodiments, the processing device 140 may evenly divide the surface profile into the subsections along a reference direction. Lengths of the subsections along the reference direction may be the same. For brevity, a length of a subsection along the reference direction may be referred to as a length of the subsection. As shown in FIG. 7, the Z-direction may be the longitudinal direction of the scanning tunnel of the medical imaging device. The processing device 140 may evenly divide the surface profile 700 into 8 subsections. A length of each of the 8 subsections along the Z-direction is Δz. In some embodiments, the lengths of the subsections may be relatively small, so that each of the subsections may be substantially as a straight line, which may facilitate the determination of correction factors corresponding to the subsections described in operation 606. In some embodiments, the processing device 140 may divide the surface profile into the subsections with different lengths based on curvatures of different portions of the surface profile. For example, a portion of the surface profile having a larger curvature may be divided into more subsections having a shorter length than a portion of the surface profile having a smaller curvature. In this way, the generated subsections may be substantially as straight lines, and the amount (or number) of the subsections may be reduced, thereby reducing an amount of subsequent calculation and improving the efficiency of the correction of the respiratory amplitude of the subject.

In 606, for each of the plurality of subsections, the processing device 140 (e.g., the correction module 230) may determine a correction factor corresponding to the subsection.

In some embodiments, a correction factor corresponding to a subsection may reflect a relationship between the respiratory amplitude collected by the respiratory motion detector and a respiratory amplitude of the respiratory motion of the subject along the standard direction. For example, the correction factor corresponding to the subsection may be configured to transform the respiratory amplitude of the subsection to a respiratory amplitude in the standard direction.

In some embodiments, the processing device 140 may obtain an installation angle of the respiratory motion detector relative to a reference direction (e.g., the longitudinal direction of the scanning tunnel of the medical imaging device, that is, the Z-direction). The processing device 140 may determine an included angle between the subsection and the reference direction. In some embodiments, the included angle between the subsection and the reference direction refers to an included angle between a straight line in which the subsection substantially is located and the reference direction. For example, the processing device 140 may obtain multiple points on the subsection. The processing device 140 may further perform a line fitting to the multiple points to obtain a straight line. The processing device 140 may designate an included angle between the straight line and the reference direction as the included angle between the subsection and the reference direction.

The processing device 140 may determine the correction factor corresponding to the subsection based on the installation angle and the included angle. Merely by way of example, FIG. 8 is a schematic diagram illustrating an exemplary installation angle and an exemplary included angle according to some embodiments of the present disclosure. As shown in FIG. 8, a respiratory motion detector 820 is configured to collect a respiratory signal of a subject by emitting detecting signals toward the subject. A subsection 810 of a surface profile of the subject is represented by a line fitting the subsection 810, α denotes an installation angle of the respiratory motion detector 820 relative to the Z direction (i.e., an exemplary reference direction), and β denotes an included angle between the subsection 810 and the reference direction Z. The processing device 140 may determine the correction factor of the subsection 810 based on the installation angle α and the included angle β. Merely by way of example, a relationship between a respiratory amplitude of the subsection 810 collected by the respiratory motion detector 820 and a respiratory amplitude of the subsection 810 in a normal direction of the subsection 810 may be determined according to Equation (1) as below:

$$Δ x = \frac{Δ r}{cos ( α - β )} , \qquad ( 1 )$$

where, Δx denotes the respiratory amplitude of the subsection 810 in the normal direction normal of the subsection 810, Δr denotes the respiratory amplitude of the subsection 810 collected by the respiratory motion detector 820, and

$$\frac{1}{cos ( α - β )}$$

denotes the correction factor corresponding to the subsection 810. As shown in Equation (1), the correction factor of the subsection 810 may be used to transform the respiratory amplitude of the subsection 810 collected by the respiratory motion detector 820 to the respiratory amplitude of the subsection in the normal direction of the subsection 810.

In 608, the processing device 140 (e.g., the correction module 230) may correct, based on the plurality of correction factors corresponding to the plurality of subsections, the respiratory amplitude of the subject.

In some embodiments, the processing device 140 may determine a total correction factor corresponding to the surface profile of the target region based on the correction factors corresponding to the subsections. For example, the processing device 140 may determine the total correction factor corresponding to the surface profile of the target region according to Equation (2) as below:

$${C}_{total} = {\sum }_{i = 1}^{n} \frac{1}{cos ( α - {β}_{i} )} , \qquad ( 2 )$$

where, Cₜₒₜₐₗ denotes the total correction factor corresponding to the surface profile of the target region, i denotes the ith subsection,

$$\frac{1}{cos ( α - {β}_{i} )}$$

denotes the correction factor corresponding to the ith subsection, and n denotes an amount of the subsections.

In some embodiments, if the surface profile is evenly divided into the subsections along the reference direction, the processing device 140 may determine the total correction factor corresponding to the surface profile of the target region according to Equation (3) as below:

$${C}_{total} = {\sum }_{i = 1}^{\frac{{FOV}_{Ra dar}}{Δ L}} \frac{1}{cos ( α - {β}_{i} )} , \qquad ( 3 )$$

where, ΔL denotes a length of each of the subsections along the reference direction, and FOV_{Radar} denote a length of the FOV of the respiratory motion detector along the reference direction.

Further, the processing device 140 may correct the respiratory amplitude of the subject based on the total correction factor. For example, the processing device 140 may correct the respiratory amplitude of the subject according to Equation (4) as below:

$${Resp}_{real} = {Resp}_{Radar} \cdot {C}_{total} , \qquad ( 4 )$$

where, Respᵣₑₐₗ denotes the corrected respiratory amplitude of the subject, and Resp_{Radar} denotes the respiratory amplitude of the subject collected by the respiratory motion detector.

As described elsewhere in this disclosure, according to the conventional respiratory motion monitoring approaches, the respiratory signal collected by the respiratory motion detector is directly used in subsequent scan data processing. According some embodiments of the present disclosure, the respiratory amplitude of the subject may be corrected based on the surface information of the target region of the subject, which may obtain more accurate respiratory amplitude of the subject. In some embodiments, respiratory amplitudes of the subject at multiple time points during the medical scan may be corrected. The corrected respiratory amplitudes corresponding to different time points may reflect the intensities of the respiratory motion along the standard direction. In such cases, the corrected respiratory amplitudes may be comparable and accurate, and the effect of the change in the relative position of the respiratory motion detector and the body surface of the subject may be reduced or eliminated. Hence, the subsequent scan data processing based on the corrected respiratory amplitude of the subject, may be improved, thereby improving the imaging quality of the medical scan by reducing or eliminating, for example, respiratory motion-induced artifacts in a resulting image.

In some embodiments, during a medical scan of a subject, the subject needs to maintain a preset status (e.g., a still status without rigid body motion, a preset physiological motion status, etc.), so that accurate scan data may be obtained. However, due to a long scanning time or other reasons, the status of the subject may change, which reduces the accuracy of the obtained scan data, or prolongs the scanning time, and reduces the efficiency of the medical scan. For example, during a medical scan of a child, it is difficult for the child to maintain the preset status due to a long scanning time or other reasons, thereby reducing the accuracy of the obtained scan data, or prolonging the scanning time, and reducing the efficiency of the medical scan. In order to solve the above problems, the present disclosure provides systems and methods for helping a subject to maintain a preset status during a medical scan of the subject.

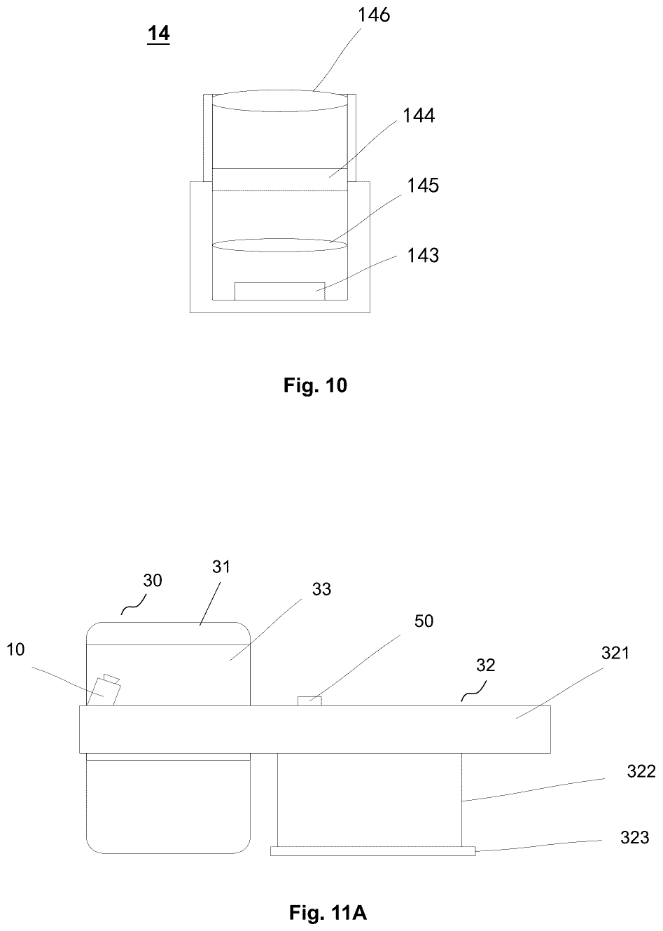

FIG. 9 is a schematic diagram illustrating an exemplary medical imaging system 900 according to some embodiments of the present disclosure. FIG. 10 is a schematic diagram illustrating an exemplary projection component 14 according to some embodiments of the present disclosure. In some embodiments, the medical imaging system 900 may be an exemplary embodiment of the medical imaging system 100.

As shown in FIG. 9, the medical imaging system 900 may include a display device 10, a control device 20, and a medical imaging device 30.

The medical imaging device 30 may be configured to scan a subject (or a part of the subject) to acquire medical image data associated with the subject. In some embodiments, the medical imaging device 30 may be similar to or the same as the medical imaging device 110. The display device 10 may be configured to display information (e.g., a video) to the subject during the medical scan, wherein the displayed information may be used to attract the subject's attention and help the subject to maintain the preset status. The control device 20 may be configured to control the display content and/or the display manner of the display device, for example, based on motion status of the subject during the medical scan.

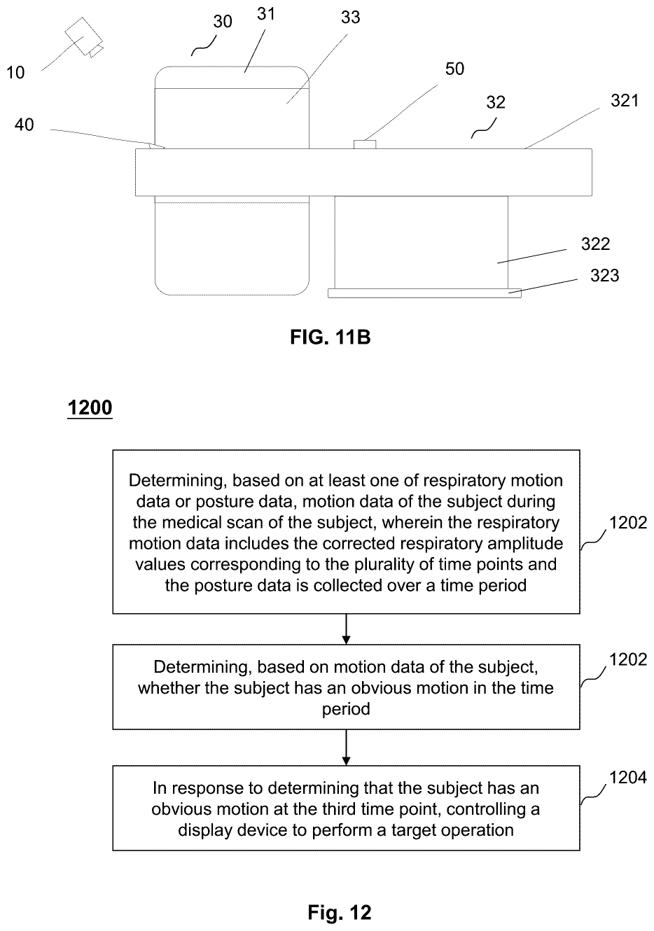

In some embodiments, the medical imaging device 30 may be electrically connected to the control device 20. The control device 20 may generate a determination signal according to a motion signal relating to the subject and send the determination signal to the medical imaging device 30. The medical imaging device 30 may determine whether scan data acquired when the subject is in the preset status satisfies requirements (e.g., the amount of the scan data exceeds a certain threshold) according to the determination signal. The medical imaging device 30 may further generate a feedback signal and send the feedback signal to the control device 20 according to the determination result. Then, the control device 20 may generate a control signal according to the feedback signal to control the display device 10 to perform a target operation. For example, in response to determining that the scan data acquired when the subject is in the preset status does not satisfy requirements, the display device 10 may display information reminding the subject to maintain the preset status, and then display preset information to attract the subject's attention until the scan data acquired by the medical imaging device 30 satisfy the requirements. More descriptions for the medical imaging device 30 may be found elsewhere in the present disclosure (e.g., FIG. 11A and FIG. 11B and the descriptions thereof).

In some embodiments, the control device 20 may be configured to detect a motion of the subject, and control the display device 10 to perform a target operation according to the detection result. For example, the target operation may include stopping displaying, changing the display content, etc. If the detection result indicates that the motion of the subject exceeds a motion threshold, the control device 20 may send a control signal to the display device 10 to control the display device 10 to perform the target operation. More descriptions for the controlling a display device to perform a target operation may be found elsewhere in the present disclosure (e.g., FIG. 12 and the descriptions thereof).

In some embodiments, the control device 20 may include a detection component 21 and a control component 22. The detection component 21 may be electrically connected with the control component 22, and the control component 22 may be electrically connected with the display device 10. The detection component 21 may be configured to detect a motion of the subject that is scanned and generate a motion signal relating to the detected motion. The control component 22 may receive the motion signal and generate a control signal according to the motion signal. The control signal may be sent to the display device 10 to control the display device 10 perform the target operation.

In some embodiments, the detection component 21 may include a plurality of varistors, a battery, and a first detection chip. The plurality of varistors may be arranged on a scanning table of the media imaging device 30 and electrically connected to the battery and the first detection chip. When the subject that is scanned moves on the scanning table, resistances of the plurality of varistors may change, which in turn causes a change in the current of the first detection chip and trigger the first detection chip to send the motion signal to the control component 22.

In some embodiments, the detection component 21 may include an image acquisition device (e.g., the image acquisition device 130 described in FIG. 1), an identification module, and a second detection chip. The image acquisition device and the second detection chip may be electrically connected with the identification module. The image acquisition device may periodically or irregularly acquire images of the subject, and send the images to the identification module. The identification module may recognize and compare the images, and determine motion data (e.g., a motion amplitude) of the subject. Based on the motion data, the identification module may determine whether the detection chip needs to be triggered to send the motion signal to the control component 22. For example, if the motion amplitude of the subject exceeds an amplitude threshold, the detection chip may be triggered to send the motion signal to the control component 22, and the control component 22 may record the motion data. Exemplary motion data may include a time when the subject starts moving, a time when the subject stops moving, a displacement, or the like, of the subject.

The display device 10 may be configured to display information (e.g., images, videos, etc.) received from other components (e.g., a storage device) of the medical imaging system 900 or stored in its internal storage device. For example, during a medical scan of the subject performed by the medical imaging device 30, the display device 10 may display preset information to attract the subject's attention, so that the subject can maintain the preset status (e.g., a still status, a breath holding status). In some embodiments, the display device 10 may be similar to or the same as the display device 150. For illustration purposes, a projector is described hereinafter as an example of the display device 10. The projector may be configured to project information within an FOV of the subject that is scanned.

In some embodiments, as shown in FIG. 9, the projector may include a signal receiver 11, a processor 12, a storage device 13, and a projection component 14. The signal receiver 11, the storage device 13, and the projection component 14 may be electrically connected to the processor 12, respectively. The signal receiver 11 may be electrically connected to the control device 20 and receive a control signal from the control device 20. The signal receiver 11 may send the received control signal to the processor 12, and the processor 12 may retrieve information to be projected from the storage device 13 according to the control signal. The retrieved information to be projected may be sent to the projection component 14 for projection.

As shown in FIGS. 9 and 10, the projection component 14 may include a beam projection control component 141, a light source driver 142, a light source 143, an image panel 144, a projection lens 145, and an illumination lens 146. The beam projection control component 141 may be electrically connected to the processor 12. The beam projection control component 141 may receive data sent by the processor 12 and convert the received data into a signal (e.g., a video signal) for beam projection. The signal for beam projection may be sent to the image panel 144. The image panel 144 may include a transmissive liquid crystal display (LCD) panel, a reflective digital micromirror device (DMD) panel, or the like. The beam projection control component 141 may also send a light source driving signal corresponding to the signal for beam projection to the light source driver 142. The image panel 144 may generate an image based on the signal for beam projection. When the light source driver 142 drives the light source 143 according to the received light source driving signal, a light beam emitted by the light source 143 may be irradiated onto the image panel 144 through the illumination lens 146, so that the image panel 144 emits a light beam and the light beam is projected through the projection lens 145. The projection lens 145 may focus manually or automatically so that the projected light beam displays an image or a video.

In some embodiments, the medical imaging system 900 may include a controller (not shown in FIG. 9). The controller may communicate with the beam projection control component 141. The subject or a user (e.g., a doctor) may operate the controller to generate a control signal for controlling the display device 10.

FIG. 11A and FIG. 11B are schematic diagrams illustrating the display device 10 and the medical imaging device 30 of the medical imaging system 900 in FIG. 9 according to some embodiments of the present disclosure.

In some embodiments, the medical imaging device 30 may include a processing assembly (e.g., a processor) and a scanning assembly. The scanning assembly may be configured to scan a subject to acquire image data. The processing assembly may be configured to process data and/or information obtained from one or more components (e.g., the scanning assembly of the medical imaging device 30, the control device 20, etc.) of the medical imaging system 900. In some embodiments, the processing assembly may be independent from the medical imaging device. For example, the processing assembly may be part of the control device 20.

In some embodiments, the processing assembly may include a selection module. The selection module may be electrically connected to a control component of a control device (e.g., the control component 22 of the control device 20). The selection module may obtain motion information of the subject collected by the control component and generate a first corresponding relationship between the motion information of the subject and the image data. For example, a first corresponding relationship may be established between a set of image data and a set of motion data collected at the same time. The selection module may be configured to divide the image data into a plurality of image data segments. Each image data segment may be collected by the medical imaging device 30 from a time point when the subject starts to move. The selection module may select the image data segment with the smallest motion amplitude and the longest duration. The image data segment with the smallest motion amplitude and the longest duration may be used for reconstructing a scan image of the subject.

In some embodiments, the medical imaging device 30 may include a correction module. The correction module may be electrically connected with the control component. The correction module may obtain the motion information collected by the control component and the image data collected by the scanning assembly, and determine a portion of the image data that is collected when the subject moves. The correction module may further perform a coordinate correction on the determined portion of the image data based on the motion information to reconstruct the scan image of the subject.

In some embodiments, the medical imaging device 30 may include an interception module. The interception module may be electrically connected with the control component. The interception module may obtain the motion information collected by the control component and generate a second corresponding relationship between the motion information of the subject and the image data. For example, a second corresponding relationship may be established between a set of image data and a set of motion data collected at the same time. The interception module may divide the image data into a plurality of image data segments according to at least one time point when the subject starts moving and at least one time point when the subject stops moving, and intercept an image data segment with no motion and the longest duration. The image data segment with no motion and the longest duration may be used for reconstructing the scan image of the subject.

In some embodiments, the medical imaging device 30 may include a gantry 31 and a scanning table 32. The gantry 31 may be used to accommodate some components of medical imaging device 30. The gantry 31 may be in a shape of a hollow cylinder, and a scanning tunnel 33 may be formed inside the gantry 31. The scanning tunnel 33 may be a space for performing medical imaging or treatment of the subject. In some embodiments, the gantry 31 may be in a shape of a square barrel with a rectangular cross section. In some embodiments, the cross section of the gantry 31 may have another shape, such as a rhombus, a hexagon, or other polygons. In some embodiments, the scanning tunnel 33 may pass through both ends of the gantry 31 along an axis direction of the gantry 31. In some embodiments, the scanning tunnel 33 may extend along the axial direction of the gantry 31, but only penetrate one end of the gantry 31, and the other end of the gantry 31 may have a closed structure.

In some embodiments, the gantry 31 may have a closed structure in a circumferential direction of the gantry 31, and a cross-section of the circumferential direction of the gantry 31 may be a closed ring. In some embodiments, the gantry 31 may have a structure that is not completely closed in the circumferential direction of the gantry 31. In some embodiments, the gantry 31 may have a completely open structure, such as a C-arm structure. The C-arm structure may include an X-ray tube and an X-ray detector opposite to the X-ray tube. A space between the X-ray tube and the X-ray detector may be form the scanning tunnel 33 of the medical imaging device 30.

The scanning table 32 may be configured to support the subject. In some embodiments, the scanning table 32 may include a table top 321 for supporting the subject, a displacement mechanism 322, and a base 323. The displacement mechanism 322 may be fixed on the base 323, and the displacement mechanism 322 may have a movable end connected with the table top 321. The scanning tunnel 33 may be located on a movement path of the table top 321, and the movable end of the displacement mechanism 322 may drive the table top 321 to move relative to the base 323 (e.g., move into or out from the scanning tunnel 33). For example, the displacement mechanism 322 may drive the table top 321 to move the object that is located at the table top 321 into the scanning tunnel 33 to perform medical imaging or treatment on the subject. As another example, after the medical scan is completed, the displacement mechanism 322 may drive the table top 321 to move the subject out from the scanning tunnel 33.

In some embodiments, the displacement mechanism 322 may include a telescopic oil cylinder fixed on the base 323. A movable end of the telescopic oil cylinder may be fixed to the table top 321 and can be extended and retracted along a movement direction of the table top 321. The movable end of the telescopic oil cylinder telescopically may drive the table top 321 to move along the movement direction of the table top 321, so as to adjust a position of the subject on the table top 321.

In some embodiments, the displacement mechanism 322 may include a slider and a sliding track. The sliding track may be fixed on a surface of the base 323 facing the table top 321. The slider may be fixed to the table top 321 and slidably connected with the sliding track. The slider may slide along the sliding track, so that the table top 321 may be driven to move along the sliding track, and the position of the subject on the table top 321 may be adjusted.

In some embodiments, the display device 10 may be located inside or outside the scanning tunnel 33. For illustration purposes, a projector is described hereinafter as an example of the display device 10. As shown in FIG. 11A, the display device 10 may be arranged inside the scanning tunnel 33, for example, arranged on the table top 321. The display device 10 may directly project information on an inner wall of the scanning tunnel 33. In some embodiments, a projection component of the display device 10 (e.g., the projection component 14 shown in FIG. 9) may face the inner wall of scanning tunnel 33. The projection component may be arranged on a side of the table top 321 or a position close to the feet of the subject. In some embodiments, the display device 10 may be rotatably connected to the table top 321, and can stop at any position along its rotation trajectory. The display device 10 may rotate relative to the table top 321 to adjust a position of the projection information on the inner wall of the scanning tunnel 33, so that needs of different subjects may be satisfied.

As shown in FIG. 11B, the display device 10 may be arranged outside the medical imaging device 30. A reflector 40 may be disposed inside the scanning tunnel 33, for example, arranged on the table top 321. An optical reflection path may be formed among the projection component of the display device 10, the reflector 40, and the inner wall of the scanning tunnel 33. The reflector 40 may reflect the information projected by the projection component to the inner wall of the scanning tunnel 33.

In some embodiments, the reflector 40 may be rotatably connected to the table top 321, and can stop at any position along its rotation trajectory. By rotating the reflector 40 relative to the table top 321, the position of the projection information reflected to the inner wall of the scanning tunnel 33 may be adjusted, so that needs of different subjects may be satisfied.

In some embodiments, the medical imaging system 900 may also include a controller 50. The controller 50 may be arranged on the scanning table 32. During the medical scan, the subject may control the display information through the controller 50.

Merely by way of example, the displacement mechanism 322 may drive the table top 321 to move the subject on the table top 321 into the scanning tunnel 33 to perform the medical scan on the subject. During the medical scan, the display device 10 may project preset information on the inner wall of the scanning tunnel 33 to attract attention of the subject. if the control device 20 detects a motion of the subject, the control device 20 may send a control signal to control the display device 10 perform a target operation. The subject may also adjust the projected content through the controller 50, so as to ensure that the projected content matches the subject's interest. This may effectively reduce the boring feeling of the subject during the medical scan to help the subject can maintain the preset status, thereby improving the accuracy and efficiency of obtaining of the scan data.

FIG. 12 is a flowchart illustrating an exemplary process 1200 for helping a subject to maintain a preset status during a medical scan of the subject according to some embodiments of the present disclosure. In some embodiments, the process 1200 may be implemented in the medical imaging system 100 illustrated in FIG. 1 or the medical imaging system 900 illustrated in FIG. 9. For example, the process 1200 may be stored in the storage device 160 of the medical imaging system 100 as a form of instructions, and invoked and/or executed by the processing device 140 (e.g., one or more modules as illustrated in FIG. 2). In some embodiments, the process 1200 may be performed by a control device (e.g., the control device 20 described in FIG. 9) or a processing assembly as described in connection with FIG. 11A and FIG. 11B.

In 1202, the processing device 140 (e.g., the determination module 210) may determine, based on at least one of respiratory motion data or posture data, motion data of the subject during the medical scan of the subject, wherein the respiratory motion data includes the corrected respiratory amplitude values corresponding to the plurality of time points and the posture data is collected over a time period.

In some embodiments, the subject may have a rigid body motion and a physiological motion during the medical scan. For example, the rigid motion may include a translational and/or rotational motion of at least a portion (e.g., the head, a leg, a hand) of the subject. The physiological motion may include a cardiac motion, a respiratory motion, or the like, or any combination thereof.

The motion data may reflect a motion state of the subject. In some embodiments, the motion data may include posture data relating to the rigid body motion of the subject, physiological motion data relating to the physiological motion of the subject, or the like, or any combination thereof. For example, the posture data may include position data of a plurality of portions of the subject, one or more joint angles, or the like, or any combination thereof. As another example, the physiological motion data relating to the respiratory motion of the subject may include a respiratory rate, a respiratory amplitude (or displacement), a respiratory cycle, or the like, or any combination thereof.

In some embodiments, the time period may include at least one of the plurality of time points. Alternatively, the time period may not include the plurality of time points. In some embodiments, the posture data relating to the rigid body motion of the subject may be obtained by analyzing image data collected by an image acquisition device (e.g., the image acquisition device 130) over the time period. In some embodiments, respiratory motion data relating to the respiratory motion of the subject may be obtained by analyzing a respiratory signal collected by a respiratory motion detector (e.g., the respiratory motion detector 120). For example, a respiratory amplitude of the respiratory signal of the subject at a time point may be corrected in a similar manner described in the FIG. 4, so as to obtain the corrected respiratory amplitude of the respiratory signal of the subject at the time point. In some embodiments, the motion data of the subject may include motion data reflecting the motion state of the subject over a series of time points.

In 1204, the processing device 140 (e.g., the determination module 210) may determine, based on motion data of the subject, whether the subject has an obvious motion in the time period.

In some embodiments, for each of one or more time points in the time period, the processing device 140 may determine whether the subject has an obvious motion at the time point (e.g., a current time point). The following descriptions take a third time point as an example.

In some embodiments, the processing device 140 may determine whether the subject has an obvious motion at the third time point (e.g., a current time point) by determining whether an amplitude of the body rigid motion at the third time point exceeds a threshold amplitude. Merely by way of example, a difference between posture data of the subject corresponding to the third time point and posture data of the subject corresponding to a fourth time point prior than the third time point may be determined, and the amplitude of the body rigid motion at the third time point may be determined based on the difference. If the amplitude of the body rigid motion at the third time point exceeds the threshold amplitude, the processing device 140 may determine that the subject has an obvious motion at the third time point.

In some embodiments, the processing device 140 may determine whether the subject has an obvious motion at the third time point by determining whether a change of a physiological motion of the subject from the fourth time point to the third time point exceeds a preset threshold. The following descriptions take the respiratory motion of the subject as an example. The processing device 140 may determine whether a change of the respiratory motion of the subject from the fourth time point to the third time point exceeds the preset threshold based on corrected respiratory amplitude values corresponding to the third time point, the fourth time point, and optionally one or more time points between the third and fourth time points. The change of the respiratory motion from the fourth time point to the third time point may be measured by, for example, a difference between the corrected respiratory amplitude values corresponding to the third time point and the fourth time point. Merely by way of example, the processing device 140 may determine whether the difference between the corrected respiratory amplitude values corresponding to the third time point and the fourth time point exceeds the preset threshold. In response to determining that the difference between the corrected respiratory amplitude values corresponding to the third time point and the fourth time point exceeds the preset threshold, the processing device 140 may determine that the subject has an obvious motion at the third time point. In response to determining that the difference between the corrected respiratory amplitude values corresponding to the third time point and the fourth time point does not exceed the preset threshold, the processing device 140 may determine that the subject has no obvious motion at the third time point. The preset threshold may be set manually by a user (e.g., an engineer) according to an experience value or a default setting of the medical image processing system 100, or determined by the processing device 140 according to an actual need. In some embodiments, the preset thresholds corresponding to different respiratory stages may be different. For example, a preset threshold corresponding to a period of a breath-hold of the subject may be 0. As another example, a preset threshold corresponding to a period of steady breathing of the subject may exceed 0. The period of steady breathing may include a period when the subject has just taken a breath but has not yet exhaled or has just exhaled but has not yet inhaled.

In 1206, in response to determining that the subject has an obvious motion at the third time point, the processing device 140 (e.g., the control module 240) may control a display device to perform a target operation.

The display device may be configured to display information (e.g., images, videos, etc.) to the subject during the medical scan of the subject. The display device may be the display device 10 described in FIGS. 9-11B or the display device 150 described in FIG. 1.

In some embodiments, the target operation may include stopping displaying, changing the display content, etc. For example, the processing device 140 may control the display device to stopping displaying, and control another device to remind the subject to maintain the preset status by playing a voice message. As another example, the processing device 140 may control the display device to display a reminder message to remind the subject to maintain the preset status. Merely by way of example, the display device may include a projector. The projector may be configured to project a virtual character in a first status on an inside wall of a scanning tunnel of a medical imaging device (e.g., the medical imaging device 110) that performs the medical scan. The processing device 140 may control the projector to change the projected virtual character from the first status to a second status. For example, in the first status, the virtual character may keep still or moving (for example, running). The second status may be different from the first status. The second status may indicate that the status of the subject has been changed, and remind the subject to maintain the preset status. For example, if the first status is a motion status, the second status may be a still status, and in the second status, the virtual character may remain still in a certain posture, e.g., with the head bowed in tears. Merely by way of example, in the medical scan, the virtual character may keep running when the subject remains still; and if the subject moves, the virtual character may fall and cry. By displaying the virtual character and controlling the status of the virtual character based on the status of the subject, the interactivity and the playfulness of the medical scan may be improved, which helps the subject maintains the preset status.

According to some embodiments of the present disclosure, during the medical scan of the subject, when the subject has an obvious motion, the display device may be controlled to perform a target operation, which may attract the subject's attention, so that the subject can maintain the preset status, thereby improving the accuracy of the obtained scan data and the efficiency of the medical scanning.

FIG. 13 is a flowchart illustrating an exemplary process 1300 for a foreign matter detection on a subject before a medical scan of the subject according to some embodiments of the present disclosure. In some embodiments, the process 1300 may be implemented in the medical imaging system 100 illustrated in FIG. 1. For example, the process 1300 may be stored in the storage device 160 of the medical imaging system 100 as a form of instructions, and invoked and/or executed by the processing device 140 (e.g., one or more modules as illustrated in FIG. 2).

In 1302, the processing device 140 (e.g., the acquisition module 220) may obtain a scout image of the subject collected by a scout scan, the scout scan being performed on the subject before the medical scan.

As used herein, a scout image refers to an image that can provide information used to guide the planning of the medical scan. For example, the scout image may be used to locate a scanned region of the subject to be scanned in the medical scan. Merely for illustration purposes, the scout image may include a positioning box enclosing a region, and an internal anatomic structure of the region may be determined according to the positioning box and an optical image of the subject. A scan range, a scan angle, a delay time, etc., of the medical scan, may be determined according to the scout image, so that a detailed planning of the medical scan may be determined to facilitate the subsequent medical diagnosis. The subject may include an animal, a human, or a non-biological object, etc. The scanned region of the subject may include, for example, the head, the chest, the abdomen, a breast, a leg, or the like, or a portion thereof, or a combination thereof, of the subject.

The scout scan may be a CT scan, an MR scan, a PET scan, an X-ray scan, or the like, or a combination thereof. In some embodiments, the scout scan may be performed using a second medical imaging device. The second medical imaging device may be same as or different from the medical imaging device used to perform the medical scan. In some embodiments, the scout image may be obtained according to a position indicator. The position indicator may include a laser position indicator. For instance, the laser position indicator may emit laser rays to at least one portion of the subject to mark a starting position and an ending position. The second medical imaging device may perform the scout scan from the starting position to the ending position.

In some embodiments, a preliminary scout image of the subject may be acquired by the scout scan. The preliminary scout image may be preprocessed to obtain a preprocessed scout image, and the preprocessed scout image may be designated as the scout image of the subject. Exemplary preprocessing operations performed on the preliminary scout image may include a noise reduction operation, a filtering operation, a grayscale binarization operation, a normalization enhancement operation, or the like, or any combination thereof. In this way, the scout image may be relatively clearer than the original preliminary scout image, thereby improving the accuracy of foreign matter detection performed based on the scout image.

In some embodiments, the processing device 140 determine whether the scout image of the subject satisfies requirements of the foreign matter detection. For example, the processing device 140 may determine whether the scout image is a front view or a side view. In response to determining that the scout image is a front view, the processing device 140 determine that the scout image of the subject satisfies requirements of the foreign matter detection. In response to determining that the scout image is a side view, the processing device 140 determine that the scout image of the subject does not satisfy requirements of the foreign matter detection. As another example, a posture of the subject in the scout image may be determined or identified manually by a user (e.g., a doctor, an imaging specialist, a technician) or automatically by the processing device 140. The term “automatically” refers to methods and systems that analyze information and generates results with little or no direct human intervention. The processing device 140 determine whether the scout image of the subject satisfies requirements of the foreign matter detection based on the posture of the subject in the scout image. If the subject in in a curled posture and some foreign matter in the subject cannot be detected, the processing device 140 determine that the scout image of the subject does not satisfy requirements of the foreign matter detection. If the subject is in a stretched posture and foreign matter in the subject can be detected, the processing device 140 determine that the scout image of the subject satisfies requirements of the foreign matter detection.

In response to determining that the scout image of the subject satisfies requirements of the foreign matter detection, operation 1304 may be performed. In response to determining that the scout image of the subject does not satisfy requirements of the foreign matter detection, the processing device 140 may send a prompt information to a user terminal. An additional scout image of the subject may be acquired, e.g., by asking the subject to change his/her posture and re-performing a scout scan on the subject, and used for the foreign matter detection.

In 1304, the processing device 140 (e.g., the detection module 250) may perform the foreign matter detection on the scout image of the subject using at least one foreign matter detection model.

In some embodiments, the foreign matter detection may be performed to determine whether foreign matter is disposed on or within the subject, and/or determine one or more parameters (e.g., the type, the size, the location) of the foreign matter. Foreign matter disposed on or within the subject may include one or more objects that are not naturally produced or grow by the subject but is on or inside the subject. Exemplary foreign matter may include metal (e.g., a metal zipper), a pathological stone, a swallowing diagnostic apparatus, a stent, calcified foreign matter (e.g., a fish bone, a chicken bone), or the like, or any combination thereof.

In some embodiments, the foreign matter disposed on or within the subject may include one or more objects with a high Hounsfield unit (HU) value (e.g., a HU value greater than a HU value threshold) or a high CT value (e.g., a CT value greater than a CT value threshold). A HU value or a CT value of an object may relate to the density of the object and used to measure the ability of the object to attenuate X-rays.

A foreign subject detection model may be a trained model (e.g., a trained machine learning model) configured to receive the scout image of the subject as an input, and output a result of foreign matter detection (referred to as a foreign matter detection result for brevity). The foreign matter detection result may indicate whether there is foreign matter disposed on or within the subject. Optionally, if there is foreign matter disposed on or within the subject, the foreign matter detection result may further include information relating to the foreign matter, such as the size, the position, the type, or the like, or any combination thereof, of the foreign matter. In some embodiments, the types of the foreign matter may include at least non-iatrogenic foreign matter and iatrogenic foreign matter. As used herein, non-iatrogenic foreign matter refers to foreign matter that can be taken off, such as a zipper, an accessory, a needle, etc. latrogenic foreign matter refers to foreign matter introduced by a result of medical treatment, such as a denture, a pacemaker, a bone nail, a replaced bone, etc. In some embodiments, the foreign matter detection result may include a foreign matter detection image generated by marking foreign matter in the scout image of the subject. In some embodiments, the foreign matter detection result may include one or more parameters (e.g., the size, the position, the count, the type) of foreign matter in the scout image of the subject. In some embodiments, the foreign matter detection result may include text information for describing the foreign matter in the scout image of the subject.

In some embodiments, the foreign matter detection model may include a linear regression model, a ridge regression model, a support vector regression model, a support vector machine model, a decision tree model, a fully connected neural network model, a deep learning model, etc. Exemplary deep learning models may include a deep neural network (DNN) model, a convolutional neural network (CNN) model (e.g., a fully convolutional neural network (FCN) model), a recurrent neural network (RNN) model, a feature pyramid network (FPN) model, a residual network, etc. Exemplary CNN models may include a V-Net model, a SpectralNet (SN) model, a Masked Siamese Networks (MSN) model, a U-Net model, a Link-Net model, or the like, or any combination thereof.

In some embodiments, different foreign matter detection models may be used for detecting different types of foreign matter (e.g., metallic foreign matter, ceramic foreign matter, non-iatrogenic foreign matter, iatrogenic foreign matter, etc.). In some embodiments, different foreign matter detection models may be used for detecting foreign matter located at different portions of the subject (e.g., the head, the hands, etc.).

In some embodiments, a plurality of foreign matter detection models may be used. For each foreign matter detection model, the scout image of the subject or a portion of the scout image may be directly inputted into the foreign matter detection model, and the foreign matter detection model may output a preliminary foreign matter detection result. The processing device 140 may combine the preliminary foreign matter detection results output by the foreign matter detection models into the foreign matter detection result.

In some embodiments, a plurality of specific portions of the subject may be segmented from the scout image manually by a user (e.g., a doctor, an imaging specialist, a technician) by, for example, drawing a bounding box on the scout image displayed on a user interface. Alternatively, the plurality of specific portions of the subject may be segmented by the processing device 140 automatically according to an image analysis algorithm (e.g., an image segmentation algorithm). For example, the processing device 140 may perform image segmentation on the scout image using an image segmentation algorithm. Exemplary image segmentation algorithm may include a thresholding segmentation algorithm, a compression-based algorithm, an edge detection algorithm, a machine learning-based segmentation algorithm (e.g., an image semantic segmentation model such as an FCN model, a U-Net model, etc.), or the like, or any combination thereof.

For example, FIG. 14 is a schematic diagram illustrating an exemplary scout image 1400 of a patient according to some embodiments of the present disclosure. As shown in FIG. 14, a plurality of specific portions of the patient may be segmented from the scout image 1400. For example, the plurality of specific portions of the subject may include the head 1410, the chest 1420, the abdomen 1430, the pelvis 1440, and lower limbs 1450 of the patient. Each of the plurality of specific portions of the subject may be inputted into a corresponding foreign matter detection model, and the foreign matter detection model may output a preliminary foreign matter detection result. The processing device 140 may combine the plurality of preliminary foreign matter detection results into the foreign matter detection result. Some types of foreign matter can only disposed on or within in specific portions of the subject, for example, dentures can only be located in the head, and barely located in other portions of the subject. Each of the plurality of specific portions of the subject may be inputted into a corresponding foreign matter detection model, which may greatly improve the accuracy of foreign matter detection. Moreover, in this way, an amount of data processed by the foreign matter detection models can be greatly reduced, thereby improving the efficiency of the foreign matter detection.

In some embodiments, the processing device 140 may obtain the at least one foreign matter detection model from one or more components of the medical imaging system 100 (e.g., the storage device 160), or an external source via a network. For example, the at least one foreign matter detection model may be previously trained by a computing device (e.g., the processing device 140, a processing device of a vendor of the foreign matter detection model), and stored in a storage device (e.g., the storage device 160) of the medical imaging system 100. The processing device 140 may access the storage device and retrieve the at least one foreign matter detection model.

In some embodiments, a foreign matter detection model may be trained according to a supervised learning algorithm by the processing device 140 or another computing device (e.g., a computing device of a vendor of the foreign matter detection model). Merely by way of example, the processing device 140 may obtain one or more training samples and a preliminary model. Each training sample may include a sample scout image of a sample subject and a ground truth foreign matter detection result. For example, the ground truth foreign matter detection result of a training sample may include a labelled scout image generated by labelling foreign matter in the sample scout image of the training sample. As another example, the ground truth foreign matter detection result may include one or more parameters (e.g., the size, the position, the count, the type) of sample foreign matter in the sample scout image of the training sample. In some embodiments, the ground truth foreign matter detection result may be determined manually by a user or automatically by the processing device 140. For example, the sample foreign matter in the sample scout image may be identified and labelled in the sample scout image by a user (a technician) to obtain the ground truth foreign matter detection result. In some embodiments, one or more parameters (e.g., the size, the position, the count, the type) of the sample foreign matter in the sample scout image may be also annotated in the sample scout image by the user. As another example, a plurality of sample portions of the sample subject may be segmented from the sample scout image. A user may select one or more sample portions from the plurality of sample portions of the sample subject, and annotate information relating to the sample foreign matter in the selected one or more sample portions to obtain the ground truth foreign matter detection result.

The preliminary model may include one or more model parameters, such as the number (or count) of layers, the number (or count) of nodes, a loss function, or the like, or any combination thereof. Before training, the preliminary model may have one or more initial parameter values of the model parameter(s).

The training of the preliminary model may include one or more iterations to iteratively update the model parameters of the preliminary model based on the training sample(s) until a termination condition is satisfied in a certain iteration. Exemplary termination conditions may be that the value of a loss function obtained in the certain iteration is less than a threshold value, that a certain count of iterations has been performed, that the loss function converges such that the difference of the values of the loss function obtained in a previous iteration and the current iteration is within a threshold value, etc. The loss function may be used to measure a discrepancy between a foreign matter detection result predicted by the preliminary model in an iteration and the ground truth foreign matter detection result. For example, the sample scout image of each training sample may be inputted into the preliminary model, and the preliminary model may output a predicted labelled scout image of the training sample. The loss function may be used to measure a difference between the predicted labelled scout image and the ground truth labelled scout image of each training sample. Exemplary loss functions may include a focal loss function, a log loss function, a cross-entropy loss, a Dice ratio, or the like. If the termination condition is not satisfied in the current iteration, the processing device 140 may further update the preliminary model to be used in a next iteration according to, for example, a backpropagation algorithm. If the termination condition is satisfied in the current iteration, the processing device 140 may designate the preliminary model in the current iteration as the foreign matter detection model.

In 1306, the processing device 140 (e.g., the determination module 210) may determine, based on the foreign matter detection result, whether the medical scan can be started.

In some embodiments, the foreign matter detection result may indicate whether there is foreign matter disposed on or within the subject. If the foreign matter detection result indicates that there is no foreign matter disposed on or within the subject, the processing device 140 may determine that the medical scan can be started. If the foreign matter detection result indicates that there is foreign matter disposed on or within the subject, the processing device 140 may generate prompt information to prompt a user according to the foreign matter detection result.

In some embodiments, the prompt information may include a foreign matter detection image. The foreign matter detection image may be generated by marking foreign matter in the scout image or a copy image of the scout image with one or more markers. Merely by way of example, each foreign matter may be marked in the scout image using a bounding box enclosing the foreign matter. The bounding box may have the shape of a square, a rectangle, a triangle, a polygon, a circle, an ellipse, an irregular shape, or the like. For example, FIG. 15 is a schematic diagram illustrating an exemplary foreign matter detection image according to some embodiments of the present disclosure. As shown in FIG. 15, multiple foreign matter is marked in the foreign matter detection image 1500 using multiple circular bounding boxes (e.g., a bounding box 1510, a bounding box 1520). Each of the multiple circular bounding boxes may enclose one of the multiple foreign matter.

In some embodiments, the prompt information may include information relating to the foreign matter, such as the size, the position, the type, the shape, an amount, or the like, of the foreign matter. The position of the foreign matter in the scout image may be represented by coordinates of the foreign matter, the portion of the subject where the foreign matter is located at, or by labeling the foreign matter in the scout image using a bounding box, etc. The size of the foreign matter may be represented by the length, the width, and the height of the foreign matter. The type of foreign matter may include at least iatrogenic foreign matter and non-iatrogenic foreign matter.

In some embodiments, the information relating to the foreign matter may be displayed in the vicinity of the foreign matter in the form of text. For example, the information relating to the foreign matter may be directly attached to the outside of the bounding box of the foreign matter, displayed inside the bounding box, or displayed on one side of the foreign matter detection image. As another example, a button may be displayed on the bounding box. When the button is clicked, a pop-up window may pop up, and the pop-up window may display the information relating to the foreign matter. In some embodiments, the prompt information may be displayed in other forms, such as voice. For example, a voice message may be played through a voice playback device (e.g., a user terminal) to broadcast the information relating to the foreign matter. If the medical imaging system 100 includes a voice playback device, the voice message including the prompt information may be directly played through the voice playback device. If the medical imaging system 100 does not include a voice playback device, a driving signal including the prompt information may be transmitted to an interactive device, and the driving signal including the prompt information may be converted into a voice message via the interactive device. Then the converted voice message may be played to broadcast the information relating to the foreign matter. The interactive device may be a smart device such as a computer with a voice conversion function, a voice player, or the like.

In some embodiments, the prompt information may be displayed in a plurality of forms at the same time. For example, the prompt information may be displayed by a voice message and a foreign matter detection image at the same time. On the one hand, the foreign matter detection image may be used to remind users that artifact correction needs to be performed for the medical scan or the scout scan needs to be reperformed. On the other hand, the subject may be reminded to take off some foreign matter (e.g., non-iatrogenic foreign matter) through the voice message, thereby improving the efficiency of the medical scan and the count of the technician entering a medical scanning room for performing the medical scan.

In some embodiments, for each of the foreign matter, the processing device 140 may determine whether the foreign matter is non-iatrogenic foreign matter or iatrogenic foreign matter according to the information relating to the foreign matter. In response to determining that non-iatrogenic foreign matter is disposed on or within the subject, the processing device 140 may generate first prompt information for requiring the subject to take off the non-iatrogenic foreign matter.

In response to determining that iatrogenic foreign matter is disposed on or within the subject, the processing device 140 may generate second prompt information for reminding that artifact correction needs to be performed for the medical scan. For example, if metallic foreign matter is disposed on or within the subject, during an MRI scan of the subject, a relatively low magnetic field strength may be used for the MRI scan, so that the magnetic susceptibility increases with the increase of the magnetic field strength to achieve the artifact correction for the metallic foreign matter. In some embodiments, since multiple 180° polyphase pulses may correct dephasing caused by a non-uniform magnetic field, the artifact correction may be performed on the metallic foreign matter using a fast spin echo (FSE) sequence with an echo interval as short as possible. The artifact correction may be performed on the metallic foreign matter by other manners, such as reducing layer thickness, using a parallel acquisition technique, reducing image distortion within a plane, reducing image distortion between slices, etc. In some embodiments, after the second prompt information is received by a user, the user may determine whether artifact correction needs to be performed on the scan data of the subject acquired by the medical scan according to the information relating to the iatrogenic foreign matter.

In some embodiments, if non-iatrogenic foreign matter is disposed on or within the subject, after the non-iatrogenic foreign matter is taken off, the processing device 140 may determine that the medical scan can be started. if only iatrogenic foreign matter is disposed on or within the subject, the processing device 140 may directly determine that the medical scan can be started.

Conventionally, a user (e.g., an imaging specialist) may need to visual check the scout image of the subject and identify foreign matter from the scout image according to experience, which has a low efficiency and accuracy. Compared with the conventional foreign matter detection approach which involves a lot of human intervention, according to some embodiments of the present disclosure, the foreign matter detection may be implemented on the scout image of the subject using at least one foreign matter detection model, which has reduced or minimal or without user intervention, thereby improving the efficiency and accuracy of the foreign matter detection model by, e.g., reducing the workload of a user, cross-user variations, and the time needed for the foreign matter detection, and in turn improving the efficiency and accuracy of the medical scan.

It will be apparent to those skilled in the art that various changes and modifications can be made in the present disclosure without departing from the spirit and scope of the disclosure. In this manner, the present disclosure may be intended to include such modifications and variations if the modifications and variations of the present disclosure are within the scope of the appended claims and the equivalents thereof. For example, the operations of the illustrated processes 400, 600, 1200, and 1300 are intended to be illustrative. In some embodiments, the processes 400, 600, 1200, and 1300 may be accomplished with one or more additional operations not described, and/or without one or more of the operations discussed. Additionally, the order in which the operations of the processes 400, 600, 1200, and 1300 and regarding descriptions are not intended to be limiting.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “module,” “unit,” “component,” “device,” or “system.” Furthermore, aspects of the present disclosure may take the form of a computer program product embodied in one or more computer readable media having computer readable program code embodied thereon.

A computer readable signal medium may include a propagated data signal with computer readable program code embodied therein, for example, in baseband or as part of a carrier wave. Such a propagated signal may take any of a variety of forms, including electro-magnetic, optical, or the like, or any suitable combination thereof. A computer readable signal medium may be any computer readable medium that is not a computer readable storage medium and that may communicate, propagate, or transport a program for use by or in connection with an instruction execution system, apparatus, or device. Program code embodied on a computer readable signal medium may be transmitted using any appropriate medium, including wireless, wireline, optical fiber cable, RF, or the like, or any suitable combination of the foregoing.

Computer program code for carrying out operations for aspects of the present disclosure may be written in any combination of one or more programming languages, including an object oriented programming language such as Java, Scala, Smalltalk, Eiffel, JADE, Emerald, C++, C #, VB. NET, Python or the like, conventional procedural programming languages, such as the “C” programming language, Visual Basic, Fortran 2003, Perl, COBOL 2002, PHP, ABAP, dynamic programming languages such as Python, Ruby and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (Saas).

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. This method of disclosure, however, is not to be interpreted as reflecting an intention that the claimed subject matter requires more features than are expressly recited in each claim. Rather, claim subject matter lie in less than all features of a single foregoing disclosed embodiment.

In some embodiments, the numbers expressing quantities or properties used to describe and claim certain embodiments of the application are to be understood as being modified in some instances by the term “about,” “approximate,” or “substantially.” For example, “about,” “approximate,” or “substantially” may indicate a certain variation (e.g., ±1%, ±5%, ±10%, or ±20%) of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques.

Notwithstanding that the numerical ranges and parameters setting forth the broad scope of some embodiments of the application are approximations, the numerical values set forth in the specific examples are reported as precisely as practicable. In some embodiments, a classification condition used in classification or determination is provided for illustration purposes and modified according to different situations. For example, a classification condition that “a value is greater than the threshold value” may further include or exclude a condition that “the probability value is equal to the threshold value.”

1. A system, comprising:
at least one storage device including a set of instructions; and
at least one processor configured to communicate with the at least one storage device, wherein when executing the set of instructions, the at least one processor is configured to direct the system to perform operations including:
determining a respiratory amplitude of a respiratory motion of a subject during a medical scan based on a respiratory signal relating to the respiratory motion, wherein the respiratory signal is collected using a respiratory motion detector by emitting detecting signals toward a target region of the subject;
obtaining surface information of the target region; and
correcting the respiratory amplitude based on the surface information of the target region.

2. The system of claim 1, wherein the corrected respiratory amplitude reflects an intensity of the respiratory motion of the subject along a standard direction.

3. The system of claim 1, wherein the obtaining surface information of the target region includes:
acquiring, using an image acquisition device, a three-dimensional (3D) optical image of the subject; and
determining, based on the 3D optical image of the subject, the surface information of the target region.

4. The system of claim 1, wherein the correcting the respiratory amplitude based on the surface information of the target region includes:
determining, based on the surface information of the target region, a surface profile of the target region;
dividing the surface profile into a plurality of subsections,
for each of the plurality of subsections, determining a correction factor corresponding to the subsection; and
correcting, based on the plurality of correction factors corresponding to the plurality of subsections, the respiratory amplitude of the subject.

5. The system of claim 4, wherein the for each of the plurality of subsections, determining a correction factor corresponding to the subsection includes:
obtaining an installation angle of the respiratory motion detector relative to a reference direction;
determining an included angle between the subsection and the reference direction; and
determining, based on the installation angle and the included angle, the correction factor corresponding to the subsection.

6. The system of claim 1, wherein:
the determining a respiratory amplitude of a respiratory motion of a subject comprises determining a plurality of respiratory amplitudes of the respiratory motion at a plurality of time points during the medical scan based on the respiratory signal,
the obtaining surface information of the target region comprises obtaining sets of surface information of the target region, each of the sets of surface information corresponding to one of the plurality of time points, and
the correcting the respiratory amplitude comprises, for each of the plurality of time points, correcting the respiratory amplitude at the time point based on the surface information corresponding to the time point.

7. The system of claim 6, wherein the operations further comprise:
obtaining scan data of the subject collected by medical scan; and
processing the scan data of the subject based on the corrected respiratory amplitudes corresponding to the plurality of time points.

8. The system of claim 6, wherein the operations further comprise:
determining, based on at least one of respiratory motion data or posture data, motion data of the subject, wherein the respiratory motion data includes the corrected respiratory amplitude values corresponding to the plurality of time points and the posture data is collected over a time period including the plurality of time points;
determining, based on the motion data of the subject, whether the subject has an obvious motion in the time period; and
in response to determining that the subject has an obvious motion in the time period, controlling a display device to perform a target operation.

9. The system of claim 8, wherein the display device includes a projector disposed in a scanning tunnel of a medical scanner that performs the medical scan.

10. The system of claim 9, wherein the projector is configured to project a virtual character in a first status on an inside wall of the scanning tunnel, and the controlling a display device to perform a target operation includes:
controlling the projector to change the projected virtual character from the first status to a second status.

11. The system of claim 1, wherein the operations further comprise:
obtaining a scout image of the subject collected by a scout scan, the scout scan being performed on the subject before the medical scan;
performing foreign matter detection on the scout image of the subject using at least one foreign matter detection model; and
determining, based on a result of the foreign matter detection, whether the medical scan can be started.

12. The system of claim 11, wherein the operations further comprise:
in response to a result of the foreign matter detection that non-iatrogenic foreign matter is disposed on or within the subject, generating first prompt information for requiring the subject to take off the non-iatrogenic foreign matter.

13. The system of claim 11, wherein the operations further comprise:
in response to a result of the foreign matter detection that iatrogenic foreign matter is disposed on or within the subject, generating second prompt information for reminding that artifact correction needs to be performed on the medical scan.

14. A method, the method being implemented on a computing device having at least one storage device and at least one processor, the method comprising:
determining a respiratory amplitude of a respiratory motion of a subject during a medical scan based on a respiratory signal relating to the respiratory motion, wherein the respiratory signal is collected using a respiratory motion detector by emitting detecting signals toward a target region of the subject;
obtaining surface information of the target region; and
correcting the respiratory amplitude based on the surface information of the target region.

15. The method of claim 14, wherein the corrected respiratory amplitude reflects an intensity of the respiratory motion of the subject along a standard direction.

16. The method of claim 14, wherein the obtaining surface information of the target region includes:
acquiring, using an image acquisition device, a three-dimensional (3D) optical image of the subject; and
determining, based on the 3D optical image of the subject, the surface information of the target region.

17. The method of claim 14, wherein the correcting the respiratory amplitude based on the surface information of the target region includes:
determining, based on the surface information of the target region, a surface profile of the target region;
dividing the surface profile into a plurality of subsections,
for each of the plurality of subsections, determining a correction factor corresponding to the subsection; and
correcting, based on the plurality of correction factors corresponding to the plurality of subsections, the respiratory amplitude of the subject.

18. The method of claim 17, wherein the for each of the plurality of subsections, determining a correction factor corresponding to the subsection includes:
obtaining an installation angle of the respiratory motion detector relative to a reference direction;
determining an included angle between the subsection and the reference direction; and
determining, based on the installation angle and the included angle, the correction factor corresponding to the subsection.

19. The method of claim 14, wherein:
the determining a respiratory amplitude of a respiratory motion of a subject comprises determining a plurality of respiratory amplitudes of the respiratory motion at a plurality of time points during the medical scan based on the respiratory signal,
the obtaining surface information of the target region comprises obtaining sets of surface information of the target region, each of the sets of surface information corresponding to one of the plurality of time points, and
the correcting the respiratory amplitude comprises, for each of the plurality of time points, correcting the respiratory amplitude at the time point based on the surface information corresponding to the time point.

20-27. (canceled)

28. A non-transitory computer readable medium, comprising at least one set of instructions, wherein when executed by one or more processors of a computing device, the at least one set of instructions causes the computing device to perform a method, the method comprising:
determining a respiratory amplitude of a respiratory motion of a subject during a medical scan based on a respiratory signal relating to the respiratory motion, wherein the respiratory signal is collected using a respiratory motion detector by emitting detecting signals toward a target region of the subject;
obtaining surface information of the target region; and
correcting the respiratory amplitude based on the surface information of the target region.

29. (canceled)
